(19) United States
(12) Patent Application Publication
(10) Pub. No.: US 2017/0189194 A1
(43) Pub. Date: Jul. 6, 2017

(54) PATELLA RESURFACING PROSTHESIS

(71) Applicant: RevOrtho LLC, Great Neck, NY (US)

(72) Inventors: Craig Eric KLINGER, Great Neck, NY (US); Jose Bernardo TORO ARBELAEZ, Remsenburg-Speonk, NY (US); David Leonard HELFET, New York, NY (US)

(21) Appl. No.: 15/399,133

(22) Filed: Jan. 5, 2017

Related U.S. Application Data
(60) Provisional application No. 62/275,294, filed on Jan. 6, 2016.

(51) Int. Cl. A61F 2/38; A61F 2/30; A61F 2/28

(52) U.S. Cl. CPC A61F 2/3877; A61F 2/2846; A61F 2/30771; A61F 2002/30443; A61F 2002/30326; A61F 2002/30121; A61F 2002/30782

(57) ABSTRACT

A patella resurfacing prosthesis may include a patella resurfacing implant. The patella resurfacing prosthesis may include one or more threaded holes along a front aspect of the patella resurfacing implant, wherein the one or more threaded holes along the front aspect of the patella resurfacing implant may be configured to fixate the patella resurfacing implant to a patella of a user.

### RELATED CASES

This application claims the benefit of U.S. Provisional Application No. 62/275,294, filed on 6 Jan. 2016, the contents of which are all incorporated by reference.

### BACKGROUND

Implants may be used, e.g., when one or more human (or animal) body parts need replacing. For example, known patella resurfacing implants may have been developed as part of total knee arthroplasty and patellofemoral arthroplasty systems, in order to treat such problems as, e.g., osteoarthritis of the knee and specifically the patellofemoral joint. Such implants may be associated with, e.g., bone thermal necrosis, which may adversely affect the patella bone strength over time.

### BRIEF SUMMARY OF DISCLOSURE

In one example implementation, a patella resurfacing prosthesis may include but is not limited to a patella resurfacing implant. The patella resurfacing prosthesis may include one or more threaded holes along a front aspect of the patella resurfacing implant, wherein the one or more threaded holes along the front aspect of the patella resurfacing implant may be configured to fixate the patella resurfacing implant to a patella of a user.

One or more of the following example features may be included. The patella resurfacing prosthesis may include one or more trunnions, wherein the one or more threaded holes along the front aspect of the patella resurfacing implant may be within the one or more trunnions respectively. The one or more trunnions may include two or more trunnions arranged horizontally. The one or more trunnions may include two or more trunnions arranged vertically. At least one trunnion of the one or more trunnions may be a central trunnion. The one or more trunnions may be arranged with an asymmetrical bearing surface. The one or more trunnions may be arranged with a symmetrical bearing surface. The one or more trunnions may be configured to receive one or more screws with one or more washers. The one or more trunnions may be configured to receive one or more screws without one or more washers. The patella resurfacing prosthesis may include a base, wherein the one or more threaded holes along the front aspect of the patella resurfacing implant may be within the base. The base may include a metal base. The one or more threaded holes may be configured to receive one or more screws with one or more washers. The patella resurfacing prosthesis may include a plate, wherein the plate may be configured to receive one or more screws through one or more screw holes within the plate, with the screws configured to engage the threaded holes along the front aspect of the patella resurfacing implant. The patella resurfacing prosthesis may include a plate including one or more holes, the one or more holes may be configured to receive one of the one or more screws. The patella resurfacing prosthesis may include a plate, wherein the plate is configured to receive one or more screws through one or more screw holes within the plate, with the screws configured to engage the threaded holes along the front aspect of the patella resurfacing implant. The patella resurfacing prosthesis may include one or more channels configured to receive bone cement. The patella resurfacing prosthesis may include a polyethylene segment. The patella resurfacing prosthesis may include one or more side-plate holes along a periphery of the patella resurfacing implant. The patella resurfacing prosthesis may include a side-plate configured to receive one or more screws through the one or more side-plate holes from a periphery of the patella resurfacing implant. A bearing surface of the patella resurfacing implant may include an asymmetrical bearing surface. A bearing surface of the patella resurfacing implant may include a symmetrical bearing surface. At least a portion of the patella resurfacing implant may include a surface that is at least one of porous, textured, granular, and beaded. The one or more threaded holes may be arranged along with an asymmetrical bearing surface. The one or more threaded holes may be arranged along with a symmetrical bearing surface.

The details of one or more example implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

### BRIEF DESCRIPTION OF THE DRAWINGS

FIG. 1A is a front view from anterior of a human left knee including a distal femur, patella, proximal tibia, and proximal fibula according to one or more example implementations of the disclosure;

FIG. 1B is a left view illustrating a human left knee including a distal femur, patella, proximal tibia, and proximal fibula according to one or more example implementations of the disclosure;

FIG. 1C is a back view of a human left patella illustrating the patella articular surface and extraarticular segment according to one or more example implementations of the disclosure;

FIG. 1D is a top view of a human left patella with a reference line for surgical preparation for a patella resurfacing prosthesis according to one or more example implementations of the disclosure;

FIG. 1E is a top view of the remaining anterior aspect of a human left patella following a vertically-oriented bone cut for a patella resurfacing prosthesis according to one or more example implementations of the disclosure;

FIG. 1F is a left view of a human left patella with a reference line for surgical preparation for a patella resurfacing prosthesis according to one or more example implementations of the disclosure;

FIG. 1G is a left view of the remaining anterior aspect of a human left patella following a vertically-oriented bone cut for a patella resurfacing prosthesis according to one or more example implementations of the disclosure;

FIG. 2A is a front view illustrating a horizontal two-trunnion symmetrical patella resurfacing prosthesis with a symmetrical bearing surface according to one or more example implementations of the disclosure;

FIG. 2B is a back view illustrating the horizontal two-trunnion symmetrical patella resurfacing prosthesis with a symmetrical bearing surface according to one or more example implementations of the disclosure;

FIG. 2C is a left view illustrating the horizontal two-trunnion symmetrical patella resurfacing prosthesis with a symmetrical bearing surface according to one or more example implementations of the disclosure;

FIG. 2D is a top view illustrating the horizontal two-trunnion symmetrical patella resurfacing prosthesis with a symmetrical bearing surface according to one or more example implementations of the disclosure;

FIG. 2E is a top view illustrating the horizontal two-trunnion symmetrical patella resurfacing prosthesis with a symmetrical bearing surface implanted into a prepared left human patella with two screws and two washers according to one or more example implementations of the disclosure;

FIG. 2F is a front view illustrating the horizontal two-trunnion symmetrical patella resurfacing prosthesis with a symmetrical bearing surface implanted into a prepared left human patella with two screws and two washers according to one or more example implementations of the disclosure;

FIG. 2G is a left view illustrating the horizontal two-trunnion symmetrical patella resurfacing prosthesis with a symmetrical bearing surface implanted into a prepared left human patella with two screws and two washers according to one or more example implementations of the disclosure;

FIG. 2H is a front view illustrating horizontal two-trunnion symmetrical patella resurfacing prosthesis with a symmetrical bearing surface implanted into a prepared left human patella with two screws without washers according to one or more example implementations of the disclosure;

FIG. 2I is a left view illustrating the horizontal two-trunnion symmetrical patella resurfacing prosthesis with a symmetrical bearing surface implanted into a prepared left human patella with two screws without washers according to one or more example implementations of the disclosure;

FIG. 3A is a front view illustrating a vertical two-trunnion symmetrical patella resurfacing prosthesis with a symmetrical bearing surface according to one or more example implementations of the disclosure;

FIG. 3B is a back view illustrating the vertical two-trunnion symmetrical patella resurfacing prosthesis with a symmetrical bearing surface according to one or more example implementations of the disclosure;

FIG. 3C is a left view illustrating the vertical two-trunnion symmetrical patella resurfacing prosthesis with a symmetrical bearing surface according to one or more example implementations of the disclosure;

FIG. 3D is a top view illustrating the vertical two-trunnion symmetrical patella resurfacing prosthesis with a symmetrical bearing surface according to one or more example implementations of the disclosure;

FIG. 3E is a top view illustrating the vertical two-trunnion symmetrical patella resurfacing prosthesis with a symmetrical bearing surface implanted into a prepared left human patella with two screws and two washers according to one or more example implementations of the disclosure;

FIG. 3F is a front view illustrating the vertical two-trunnion symmetrical patella resurfacing prosthesis with a symmetrical bearing surface implanted into a prepared left human patella with two screws and two washers according to one or more example implementations of the disclosure;

FIG. 3G is a left view illustrating the vertical two-trunnion symmetrical patella resurfacing prosthesis with a symmetrical bearing surface implanted into a prepared left human patella with two screws and two washers according to one or more example implementations of the disclosure;

FIG. 3H is a front view illustrating the vertical two-trunnion symmetrical patella resurfacing prosthesis with a symmetrical bearing surface implanted into a prepared left human patella with two screws without washers according to one or more example implementations of the disclosure;

FIG. 3I is a left view illustrating the vertical two-trunnion symmetrical patella resurfacing prosthesis with a symmetrical bearing surface implanted into a prepared left human patella with two screws without washers according to one or more example implementations of the disclosure;

FIG. 4A is a front view illustrating a four-trunnion symmetrical patella resurfacing prosthesis including four trunnions with a symmetrical bearing surface according to one or more example implementations of the disclosure;

FIG. 4B is a back view illustrating the four-trunnion symmetrical patella resurfacing prosthesis including four trunnions with a symmetrical bearing surface according to one or more example implementations of the disclosure;

FIG. 4C is a left view illustrating the four-trunnion symmetrical patella resurfacing prosthesis including four trunnions with a symmetrical bearing surface according to one or more example implementations of the disclosure;

FIG. 4D is a top view illustrating the four-trunnion symmetrical patella resurfacing prosthesis including four trunnions with a symmetrical bearing surface according to one or more example implementations of the disclosure;

FIG. 4E is a top view illustrating the four-trunnion symmetrical patella resurfacing prosthesis including four trunnions with a symmetrical bearing surface implanted into a prepared left human patella with four screws and four washers according to one or more example implementations of the disclosure;

FIG. 4F is a front view illustrating the four-trunnion symmetrical patella resurfacing prosthesis including four trunnions with a symmetrical bearing surface implanted into a prepared left human patella with four screws and four washers according to one or more example implementations of the disclosure;

FIG. 4G is a left view illustrating the four-trunnion symmetrical patella resurfacing prosthesis including four trunnions with a symmetrical bearing surface implanted into a prepared left human patella with four screws and four washers according to one or more example implementations of the disclosure;

FIG. 4H is a front view illustrating the four-trunnion symmetrical patella resurfacing prosthesis including four trunnions with a symmetrical bearing surface implanted into a prepared left human patella with four screws without washers according to one or more example implementations of the disclosure;

FIG. 4I is a left view illustrating the four-trunnion symmetrical patella resurfacing prosthesis including four trunnions with a symmetrical bearing surface implanted into a prepared left human patella with four screws without washers according to one or more example implementations of the disclosure;

FIG. 5A is a front view illustrating a one-trunnion asymmetrical patella resurfacing prosthesis including one central trunnion with an asymmetrical bearing surface according to one or more example implementations of the disclosure;

FIG. 5B is a back view illustrating the one-trunnion asymmetrical patella resurfacing prosthesis including one central trunnion with an asymmetrical bearing surface according to one or more example implementations of the disclosure;

FIG. 5C is a left view illustrating the one-trunnion asymmetrical patella resurfacing prosthesis including one central trunnion with an asymmetrical bearing surface according to one or more example implementations of the disclosure;

FIG. 5D is a top view illustrating the one-trunnion asymmetrical patella resurfacing prosthesis including one central trunnion with an asymmetrical bearing surface according to one or more example implementations of the disclosure;

FIG. 5E is a top view illustrating the one-trunnion asymmetrical patella resurfacing prosthesis including one central trunnion with an asymmetrical bearing surface implanted into a prepared left human patella with one screw and one washer according to one or more example implementations of the disclosure;

FIG. 5F is a front view illustrating the one-trunnion asymmetrical patella resurfacing prosthesis including one central trunnion with an asymmetrical bearing surface implanted into a prepared left human patella with one screw and one washer according to one or more example implementations of the disclosure;

FIG. 5G is a left view illustrating the one-trunnion asymmetrical patella resurfacing prosthesis including one central trunnion with an asymmetrical bearing surface implanted into a prepared left human patella with one screw and one washer according to one or more example implementations of the disclosure;

FIG. 5H is a front view illustrating the one-trunnion asymmetrical patella resurfacing prosthesis including one central trunnion with an asymmetrical bearing surface implanted into a prepared left human patella with one screw without a washer according to one or more example implementations of the disclosure;

FIG. 5I is a left view illustrating the one-trunnion asymmetrical patella resurfacing prosthesis including one central trunnion with an asymmetrical bearing surface implanted into a prepared left human patella with one screw without a washer according to one or more example implementations of the disclosure;

FIG. 6A is a front view illustrating a one-trunnion channeled asymmetrical patella resurfacing prosthesis including one central trunnion, channels for bone cement, with an asymmetrical bearing surface according to one or more example implementations of the disclosure;

FIG. 6B is a back view illustrating the one-trunnion channeled asymmetrical patella resurfacing prosthesis including one central trunnion, channels for bone cement, with an asymmetrical bearing surface according to one or more example implementations of the disclosure;

FIG. 6C is a left view illustrating the one-trunnion channeled asymmetrical patella resurfacing prosthesis including one central trunnion, channels for bone cement, with an asymmetrical bearing surface according to one or more example implementations of the disclosure;

FIG. 6D is a top view illustrating the one-trunnion channeled asymmetrical patella resurfacing prosthesis including one central trunnion, channels for bone cement, with an asymmetrical bearing surface according to one or more example implementations of the disclosure;

FIG. 6E is a top view illustrating the one-trunnion channeled asymmetrical patella resurfacing prosthesis including one central trunnion, channels for bone cement, with an asymmetrical bearing surface implanted into a prepared left human patella with one screw and one washer according to one or more example implementations of the disclosure;

FIG. 6F is a front view illustrating the one-trunnion channeled asymmetrical patella resurfacing prosthesis including one central trunnion, channels for bone cement, with an asymmetrical bearing surface implanted into a prepared left human patella with one screws and one washer according to one or more example implementations of the disclosure;

FIG. 6G is a left view illustrating the one-trunnion channeled asymmetrical patella resurfacing prosthesis including one central trunnion, channels for bone cement, with an asymmetrical bearing surface implanted into a prepared left human patella with one screw and one washer according to one or more example implementations of the disclosure;

FIG. 6H is a front view illustrating the one-trunnion channeled asymmetrical patella resurfacing prosthesis including one central trunnion, channels for bone cement, with an asymmetrical bearing surface implanted into a prepared left human patella with one screw without a washer according to one or more example implementations of the disclosure;

FIG. 6I is a left view illustrating the one-trunnion channeled asymmetrical patella resurfacing prosthesis including one central trunnion, channels for bone cement, with an asymmetrical bearing surface implanted into a prepared left human patella with one screw without a washer according to one or more example implementations of the disclosure;

FIG. 7A is a front view illustrating a horizontal two-trunnion asymmetrical patella resurfacing prosthesis including two horizontally-arranged trunnions with an asymmetrical bearing surface according to one or more example implementations of the disclosure;

FIG. 7B is a back view illustrating horizontal two-trunnion asymmetrical patella resurfacing prosthesis including two horizontally-arranged trunnions with an asymmetrical bearing surface according to one or more example implementations of the disclosure;

FIG. 7C is a left view illustrating the horizontal two-trunnion asymmetrical patella resurfacing prosthesis including two horizontally-arranged trunnions with an asymmetrical bearing surface according to one or more example implementations of the disclosure;

FIG. 7D is a top view illustrating the horizontal two-trunnion asymmetrical patella resurfacing prosthesis including two horizontally-arranged trunnions with an asymmetrical bearing surface according to one or more example implementations of the disclosure;

FIG. 7E is a top view illustrating the horizontal two-trunnion asymmetrical patella resurfacing prosthesis including two horizontally-arranged trunnions with an asymmetrical bearing surface implanted into a prepared left human patella with two screws and two washers according to one or more example implementations of the disclosure;

FIG. 7F is a front view illustrating the horizontal two-trunnion asymmetrical patella resurfacing prosthesis including two horizontally-arranged trunnions with an asymmetrical bearing surface implanted into a prepared left human patella with two screws and two washers according to one or more example implementations of the disclosure;

FIG. 7G is a left view illustrating the horizontal two-trunnion asymmetrical patella resurfacing prosthesis including two horizontally-arranged trunnions with an asymmetrical bearing surface implanted into a prepared left human patella with two screws and two washers according to one or more example implementations of the disclosure;

FIG. 7H is a front view illustrating the horizontal two-trunnion asymmetrical patella resurfacing prosthesis including two horizontally-arranged trunnions with an asymmetrical bearing surface implanted into a prepared left human patella with two screws without washers according to one or more example implementations of the disclosure;

FIG. 7I is a left view illustrating the horizontal two-trunnion asymmetrical patella resurfacing prosthesis including two horizontally-arranged trunnions with an asymmetrical bearing surface implanted into a prepared left human patella with two screws without washers according to one or more example implementations of the disclosure;

FIG. 8A is a front view illustrating a flush horizontal two-trunnion asymmetrical patella resurfacing prosthesis including two horizontally-arranged trunnions with an asymmetrical bearing surface according to one or more example implementations of the disclosure;

FIG. 8B is a back view illustrating the flush horizontal two-trunnion asymmetrical patella resurfacing prosthesis including two horizontally-arranged trunnions with an asymmetrical bearing surface according to one or more example implementations of the disclosure;

FIG. 8C is a left view illustrating the flush horizontal two-trunnion asymmetrical patella resurfacing prosthesis including two horizontally-arranged trunnions with an asymmetrical bearing surface according to one or more example implementations of the disclosure;

FIG. 8D is a top view illustrating the flush horizontal two-trunnion asymmetrical patella resurfacing prosthesis including two horizontally-arranged trunnions with an asymmetrical bearing surface according to one or more example implementations of the disclosure;

FIG. 8E is a top view illustrating the flush horizontal two-trunnion asymmetrical patella resurfacing prosthesis including two horizontally-arranged trunnions with an asymmetrical bearing surface implanted into a prepared left human patella with two screws and two washers according to one or more example implementations of the disclosure;

FIG. 8F is a front view illustrating flush horizontal two-trunnion asymmetrical patella resurfacing prosthesis including two horizontally-arranged trunnions with an asymmetrical bearing surface implanted into a prepared left human patella with two screws and two washers according to one or more example implementations of the disclosure;

FIG. 8G is a left view illustrating the flush horizontal two-trunnion asymmetrical patella resurfacing prosthesis including two horizontally-arranged trunnions with an asymmetrical bearing surface implanted into a prepared left human patella with two screws and two washers according to one or more example implementations of the disclosure;

FIG. 8H is a front view illustrating the flush horizontal two-trunnion asymmetrical patella resurfacing prosthesis including two horizontally-arranged trunnions with an asymmetrical bearing surface implanted into a prepared left human patella with two screws without washers according to one or more example implementations of the disclosure;

FIG. 8I is a left view illustrating the flush horizontal two-trunnion asymmetrical patella resurfacing prosthesis including two horizontally-arranged trunnions with an asymmetrical bearing surface implanted into a prepared left human patella with two screws without washers according to one or more example implementations of the disclosure;

FIG. 9A is a front view illustrating a vertical two-trunnion asymmetrical patella resurfacing prosthesis including two vertically-arranged trunnions with an asymmetrical bearing surface according to one or more example implementations of the disclosure;

FIG. 9B is a back view illustrating the vertical two-trunnion asymmetrical patella resurfacing prosthesis including two vertically-arranged trunnions with an asymmetrical bearing surface according to one or more example implementations of the disclosure;

FIG. 9C is a left view illustrating the vertical two-trunnion asymmetrical patella resurfacing prosthesis including two vertically-arranged trunnions with an asymmetrical bearing surface according to one or more example implementations of the disclosure;

FIG. 9D is a top view illustrating the vertical two-trunnion asymmetrical patella resurfacing prosthesis including two vertically-arranged trunnions with an asymmetrical bearing surface according to one or more example implementations of the disclosure;

FIG. 9E is a top view illustrating the vertical two-trunnion asymmetrical patella resurfacing prosthesis including two vertically-arranged trunnions with an asymmetrical bearing surface implanted into a prepared left human patella with two screws and two washers according to one or more example implementations of the disclosure;

FIG. 9F is a front view illustrating the vertical two-trunnion asymmetrical patella resurfacing prosthesis including two vertically-arranged trunnions with an asymmetrical bearing surface implanted into a prepared left human patella with two screws and two washers according to one or more example implementations of the disclosure;

FIG. 9G is a left view illustrating the vertical two-trunnion asymmetrical patella resurfacing prosthesis including two vertically-arranged trunnions with an asymmetrical bearing surface implanted into a prepared left human patella with two screws and two washers according to one or more example implementations of the disclosure;

FIG. 9H is a front view illustrating vertical two-trunnion asymmetrical patella resurfacing prosthesis including two vertically-arranged trunnions with an asymmetrical bearing surface implanted into a prepared left human patella with two screws without washers according to one or more example implementations of the disclosure;

FIG. 9I is a left view illustrating the vertical two-trunnion asymmetrical patella resurfacing prosthesis including two vertically-arranged trunnions with an asymmetrical bearing surface implanted into a prepared left human patella with two screws without washers according to one or more example implementations of the disclosure;

FIG. 10A is a front view illustrating a four-trunnion asymmetrical patella resurfacing prosthesis including four trunnions with an asymmetrical bearing surface according to one or more example implementations of the disclosure;

FIG. 10B is a back view illustrating the four-trunnion asymmetrical patella resurfacing prosthesis including four trunnions with an asymmetrical bearing surface according to one or more example implementations of the disclosure;

FIG. 10C is a left view illustrating the four-trunnion asymmetrical patella resurfacing prosthesis including four trunnions with an asymmetrical bearing surface according to one or more example implementations of the disclosure;

FIG. 10D is a top view illustrating the four-trunnion asymmetrical patella resurfacing prosthesis including four trunnions with an asymmetrical bearing surface according to one or more example implementations of the disclosure;

FIG. 10E is a top view illustrating the four-trunnion asymmetrical patella resurfacing prosthesis including four trunnions with an asymmetrical bearing surface implanted into a prepared left human patella with four screws and four washers according to one or more example implementations of the disclosure;

FIG. 10F is a front view illustrating the four-trunnion asymmetrical patella resurfacing prosthesis including four trunnions with an asymmetrical bearing surface implanted into a prepared left human patella with four screws and four washers according to one or more example implementations of the disclosure;

FIG. 10G is a left view illustrating the four-trunnion asymmetrical patella resurfacing prosthesis including four trunnions with an asymmetrical bearing surface implanted into a prepared left human patella with four screws and four washers according to one or more example implementations of the disclosure;

FIG. 10H is a front view illustrating the four-trunnion asymmetrical patella resurfacing prosthesis including four trunnions with an asymmetrical bearing surface implanted into a prepared left human patella with four screws without washers according to one or more example implementations of the disclosure;

FIG. 10I is a left view illustrating the four-trunnion asymmetrical patella resurfacing prosthesis including four trunnions with an asymmetrical bearing surface implanted into a prepared left human patella with four screws without washers according to one or more example implementations of the disclosure;

FIG. 11A is a front view illustrating a metal horizontal two-trunnion asymmetrical patella resurfacing prosthesis including two horizontally-arranged trunnions with an asymmetrical bearing surface according to one or more example implementations of the disclosure;

FIG. 11B is a back view illustrating the metal horizontal two-trunnion asymmetrical patella resurfacing prosthesis including two horizontally-arranged trunnions with an asymmetrical bearing surface according to one or more example implementations of the disclosure;

FIG. 11C is a left view illustrating the metal horizontal two-trunnion asymmetrical patella resurfacing prosthesis including two horizontally-arranged trunnions with an asymmetrical bearing surface according to one or more example implementations of the disclosure;

FIG. 11D is a top view illustrating the metal horizontal two-trunnion asymmetrical patella resurfacing prosthesis including two horizontally-arranged trunnions with an asymmetrical bearing surface according to one or more example implementations of the disclosure;

FIG. 11E is a top view illustrating the metal horizontal two-trunnion asymmetrical patella resurfacing prosthesis including two horizontally-arranged trunnions with an asymmetrical bearing surface implanted into a prepared left human patella with two screws and two washers according to one or more example implementations of the disclosure;

FIG. 11F is a front view illustrating the metal horizontal two-trunnion asymmetrical patella resurfacing prosthesis including two horizontally-arranged trunnions with an asymmetrical bearing surface implanted into a prepared left human patella with two screws and two washers according to one or more example implementations of the disclosure;

FIG. 11G is a left view illustrating the metal horizontal two-trunnion asymmetrical patella resurfacing prosthesis including two horizontally-arranged trunnions with an asymmetrical bearing surface implanted into a prepared left human patella with two screws and two washers according to one or more example implementations of the disclosure;

FIG. 11H is a front view illustrating the metal horizontal two-trunnion asymmetrical patella resurfacing prosthesis including two horizontally-arranged trunnions with an asymmetrical bearing surface implanted into a prepared left human patella with two screws without washers according to one or more example implementations of the disclosure;

FIG. 11I is a left view illustrating the metal horizontal two-trunnion asymmetrical patella resurfacing prosthesis including two horizontally-arranged trunnions with an asymmetrical bearing surface implanted into a prepared left human patella with two screws without washers according to one or more example implementations of the disclosure;

FIG. 12A is a front view illustrating a metal two-hole asymmetrical patella resurfacing prosthesis including two horizontally-arranged threaded holes without trunnions with an asymmetrical bearing surface according to one or more example implementations of the disclosure;

FIG. 12B is a back view illustrating the metal two-hole asymmetrical patella resurfacing prosthesis including two horizontally-arranged threaded holes without trunnions with an asymmetrical bearing surface according to one or more example implementations of the disclosure;

FIG. 12C is a left view illustrating the metal two-hole asymmetrical patella resurfacing prosthesis including two horizontally-arranged threaded holes without trunnions with an asymmetrical bearing surface according to one or more example implementations of the disclosure;

FIG. 12D is a top view illustrating the metal two-hole asymmetrical patella resurfacing prosthesis including two horizontally-arranged threaded holes without trunnions with an asymmetrical bearing surface according to one or more example implementations of the disclosure;

FIG. 12E is a top view illustrating the metal two-hole asymmetrical patella resurfacing prosthesis including two horizontally-arranged threaded holes without trunnions with an asymmetrical bearing surface implanted into a prepared left human patella with two screws and two washers according to one or more example implementations of the disclosure;

FIG. 12F is a front view illustrating the metal two-hole asymmetrical patella resurfacing prosthesis including two horizontally-arranged threaded holes without trunnions with an asymmetrical bearing surface implanted into a prepared left human patella with two screws and two washers according to one or more example implementations of the disclosure;

FIG. 12G is a left view illustrating the metal two-hole asymmetrical patella resurfacing prosthesis including two horizontally-arranged threaded holes without trunnions with an asymmetrical bearing surface implanted into a prepared left human patella with two screws and two washers according to one or more example implementations of the disclosure;

FIG. 12H is a front view illustrating metal two-hole asymmetrical patella resurfacing prosthesis including two horizontally-arranged threaded holes without trunnions with an asymmetrical bearing surface implanted into a prepared left human patella with two screws without washers according to one or more example implementations of the disclosure;

FIG. 12I is a left view illustrating the metal two-hole asymmetrical patella resurfacing prosthesis including two horizontally-arranged threaded holes without trunnions with an asymmetrical bearing surface implanted into a prepared left human patella with two screws without washers according to one or more example implementations of the disclosure;

FIG. 13A is a front view illustrating a metal four-trunnion asymmetrical patella resurfacing prosthesis including four trunnions with an asymmetrical bearing surface according to one or more example implementations of the disclosure;

FIG. 13B is a back view illustrating the metal four-trunnion asymmetrical patella resurfacing prosthesis including four trunnions with an asymmetrical bearing surface according to one or more example implementations of the disclosure;

FIG. 13C is a left view illustrating the metal four-trunnion asymmetrical patella resurfacing prosthesis including four trunnions with an asymmetrical bearing surface according to one or more example implementations of the disclosure;

FIG. 13D is a top view illustrating the metal four-trunnion asymmetrical patella resurfacing prosthesis including four trunnions with an asymmetrical bearing surface according to one or more example implementations of the disclosure;

FIG. 13E is a top view illustrating the metal four-trunnion asymmetrical patella resurfacing prosthesis including four trunnions with an asymmetrical bearing surface implanted into a prepared left human patella with four screws and four washers according to one or more example implementations of the disclosure;

FIG. 13F is a front view illustrating the metal four-trunnion asymmetrical patella resurfacing prosthesis including four trunnions with an asymmetrical bearing surface implanted into a prepared left human patella with four screws and four washers according to one or more example implementations of the disclosure;

FIG. 13G is a left view illustrating the metal four-trunnion asymmetrical patella resurfacing prosthesis including four trunnions with an asymmetrical bearing surface implanted into a prepared left human patella with four screws and four washers according to one or more example implementations of the disclosure;

FIG. 13H is a front view illustrating the metal four-trunnion asymmetrical patella resurfacing prosthesis including four trunnions with an asymmetrical bearing surface implanted into a prepared left human patella with four screws without washers according to one or more example implementations of the disclosure;

FIG. 13I is a left view illustrating the metal four-trunnion asymmetrical patella resurfacing prosthesis including four trunnions with an asymmetrical bearing surface implanted into a prepared left human patella with four screws without washers according to one or more example implementations of the disclosure;

FIG. 14A is a front view illustrating a metal four-hole asymmetrical patella resurfacing prosthesis including four threaded holes without trunnions with an asymmetrical bearing surface according to one or more example implementations of the disclosure;

FIG. 14B is a back view illustrating the metal four-hole asymmetrical patella resurfacing prosthesis including four threaded holes without trunnions with an asymmetrical bearing surface according to one or more example implementations of the disclosure;

FIG. 14C is a left view illustrating the metal four-hole asymmetrical patella resurfacing prosthesis including four threaded holes without trunnions with an asymmetrical bearing surface according to one or more example implementations of the disclosure;

FIG. 14D is a top view illustrating the metal four-hole asymmetrical patella resurfacing prosthesis including four threaded holes without trunnions with an asymmetrical bearing surface according to one or more example implementations of the disclosure;

FIG. 14E is a top view illustrating the metal four-hole asymmetrical patella resurfacing prosthesis including four threaded holes without trunnions with an asymmetrical bearing surface implanted into a prepared left human patella with four screws and four washers according to one or more example implementations of the disclosure;

FIG. 14F is a front view illustrating the metal four-hole asymmetrical patella resurfacing prosthesis including four threaded holes without trunnions with an asymmetrical bearing surface implanted into a prepared left human patella with four screws and four washers according to one or more example implementations of the disclosure;

FIG. 14G is a left view illustrating the metal four-hole asymmetrical patella resurfacing prosthesis including four threaded holes without trunnions with an asymmetrical bearing surface implanted into a prepared left human patella with four screws and four washers according to one or more example implementations of the disclosure;

FIG. 14H is a front view illustrating the metal four-hole asymmetrical patella resurfacing prosthesis including four threaded holes without trunnions with an asymmetrical bearing surface implanted into a prepared left human patella with four screws without washers according to one or more example implementations of the disclosure;

FIG. 14I is a left view illustrating metal four-hole asymmetrical patella resurfacing prosthesis including four threaded holes without trunnions with an asymmetrical bearing surface implanted into a prepared left human patella with four screws without washers according to one or more example implementations of the disclosure;

FIG. 15A is a bottom view illustrating a plate with screw holes for two screws according to one or more example implementations of the disclosure;

FIG. 15B is a front view further illustrating a plate with screw holes for two screws according to one or more example implementations of the disclosure;

FIG. 15C is a left view illustrating the vertical two-trunnion symmetrical patella resurfacing prosthesis including two vertically-arranged trunnions with a symmetrical bearing surface implanted into a prepared left human patella with two screws inserted through a two-hole plate according to one or more example implementations of the disclosure;

FIG. 15D is a front view illustrating the vertical two-trunnion symmetrical patella resurfacing prosthesis including two vertically-arranged trunnions with a symmetrical bearing surface implanted into a prepared left human patella with two screws inserted through a two-hole plate according to one or more example implementations of the disclosure;

FIG. 15E is a left view illustrating the four-trunnion symmetrical patella resurfacing prosthesis including four trunnions with a symmetrical bearing surface implanted into a prepared left human patella with four screws inserted through a four-hole plate according to one or more example implementations of the disclosure;

FIG. 15F is a front view illustrating the four-trunnion symmetrical patella resurfacing prosthesis including four trunnions with a symmetrical bearing surface implanted into a prepared left human patella with four screws inserted through a four-hole plate according to one or more example implementations of the disclosure;

FIG. 15G is a top view illustrating the horizontal two-trunnion symmetrical patella resurfacing prosthesis including two horizontally-arranged trunnions with a symmetrical bearing surface implanted into a prepared left human patella with two screws inserted through a two-hole plate according to one or more example implementations of the disclosure;

FIG. 15H is a front view illustrating the horizontal two-trunnion symmetrical patella resurfacing prosthesis including two horizontally-arranged trunnions with a symmetrical bearing surface implanted into a prepared left human patella with two screws inserted through a two-hole plate according to one or more example implementations of the disclosure;

FIG. 16A is a front view illustrating a metal base of a flush horizontal two-trunnion symmetrical patella resurfacing prosthesis including two horizontally-arranged trunnions according to one or more example implementations of the disclosure;

FIG. 16B is a back view illustrating the metal base of a flush horizontal two-trunnion symmetrical patella resurfacing prosthesis including two horizontally-arranged trunnions according to one or more example implementations of the disclosure;

FIG. 16C is a left view illustrating the metal base of a flush horizontal two-trunnion symmetrical patella resurfacing prosthesis including two horizontally-arranged trunnions according to one or more example implementations of the disclosure;

FIG. 16D is a top view illustrating the metal base of a flush horizontal two-trunnion symmetrical patella resurfacing prosthesis including two horizontally-arranged trunnions according to one or more example implementations of the disclosure;

FIG. 16E is a front view illustrating a polyethylene segment of a flush horizontal two-trunnion symmetrical patella resurfacing prosthesis according to one or more example implementations of the disclosure;

FIG. 16F is a back view illustrating the polyethylene segment of a flush horizontal two-trunnion symmetrical patella resurfacing prosthesis including a symmetrical bearing surface according to one or more example implementations of the disclosure;

FIG. 16G is a left view illustrating the polyethylene segment of a flush horizontal two-trunnion symmetrical patella resurfacing prosthesis including a symmetrical bearing surface according to one or more example implementations of the disclosure;

FIG. 16H is a top view illustrating the polyethylene segment of a flush horizontal two-trunnion symmetrical patella resurfacing prosthesis including a symmetrical bearing surface according to one or more example implementations of the disclosure;

FIG. 17A is a front view illustrating a metal base of a one-trunnion asymmetrical patella resurfacing prosthesis including one central trunnion according to one or more example implementations of the disclosure;

FIG. 17B is a back view illustrating the metal base of one-trunnion asymmetrical patella resurfacing prosthesis including one central trunnion according to one or more example implementations of the disclosure;

FIG. 17C is a left view illustrating the metal base of a one-trunnion asymmetrical patella resurfacing prosthesis including one central trunnion according to one or more example implementations of the disclosure;

FIG. 17D is a top view illustrating the metal base of a one-trunnion asymmetrical patella resurfacing prosthesis including one central trunnion according to one or more example implementations of the disclosure;

FIG. 17E is a front view illustrating a polyethylene segment of a one-trunnion asymmetrical patella resurfacing prosthesis according to one or more example implementations of the disclosure;

FIG. 17F is a back view illustrating the polyethylene segment of a one-trunnion asymmetrical patella resurfacing prosthesis including an asymmetrical bearing surface according to one or more example implementations of the disclosure;

FIG. 17G is a left view illustrating the polyethylene segment of a one-trunnion asymmetrical patella resurfacing prosthesis including an asymmetrical bearing surface according to one or more example implementations of the disclosure;

FIG. 17H is a top view illustrating the polyethylene segment of a one-trunnion asymmetrical patella resurfacing prosthesis including an asymmetrical bearing surface according to one or more example implementations of the disclosure;

FIG. 18A is a front view illustrating a horizontal two-hole asymmetrical patella resurfacing prosthesis including two horizontally-arranged threaded holes with an asymmetrical bearing surface according to one or more example implementations of the disclosure;

FIG. 18B is a back view illustrating the horizontal two-hole asymmetrical patella resurfacing prosthesis including two horizontally-arranged threaded holes with an asymmetrical bearing surface according to one or more example implementations of the disclosure;

FIG. 18C is a left view illustrating the horizontal two-hole asymmetrical patella resurfacing prosthesis including two horizontally-arranged threaded holes with an asymmetrical bearing surface according to one or more example implementations of the disclosure;

FIG. 18D is a top view illustrating the horizontal two-hole asymmetrical patella resurfacing prosthesis including two horizontally-arranged threaded holes with an asymmetrical bearing surface according to one or more example implementations of the disclosure;

FIG. 18E is a top view illustrating the metal base of a horizontal two-hole asymmetrical patella resurfacing prosthesis including two horizontally-arranged threaded holes with an asymmetrical bearing surface according to one or more example implementations of the disclosure;

FIG. 18F is a front view illustrating a horizontal two-hole symmetrical patella resurfacing prosthesis including two horizontally-arranged threaded holes with a symmetrical bearing surface according to one or more example implementations of the disclosure;

FIG. 18G is a back view illustrating the horizontal two-hole symmetrical patella resurfacing prosthesis including two horizontally-arranged threaded holes with a symmetrical bearing surface according to one or more example implementations of the disclosure;

FIG. 18H is a left view illustrating the horizontal two-hole symmetrical patella resurfacing prosthesis including two horizontally-arranged threaded holes with a symmetrical bearing surface according to one or more example implementations of the disclosure;

FIG. 18I is a top view illustrating the horizontal two-hole symmetrical patella resurfacing prosthesis including two horizontally-arranged threaded holes with a symmetrical bearing surface according to one or more example implementations of the disclosure;

FIG. 18J is a top view illustrating the metal base of a horizontal two-hole symmetrical patella resurfacing prosthesis including two horizontally-arranged threaded holes with a symmetrical bearing surface according to one or more example implementations of the disclosure;

FIG. 19A is a front view illustrating a one-trunnion asymmetrical patella resurfacing prosthesis with side-plate including one central trunnion, side-plate, with an asymmetrical bearing surface according to one or more example implementations of the disclosure;

FIG. 19B is a back view illustrating the one-trunnion asymmetrical patella resurfacing prosthesis with side-plate including one central trunnion, side-plate, with an asymmetrical bearing surface according to one or more example implementations of the disclosure;

FIG. 19C is a left view illustrating the one-trunnion asymmetrical patella resurfacing prosthesis with side-plate including one central trunnion, side-plate, with an asymmetrical bearing surface according to one or more example implementations of the disclosure;

FIG. 19D is a top view illustrating the one-trunnion asymmetrical patella resurfacing prosthesis with side-plate including one central trunnion, side-plate, with an asymmetrical bearing surface according to one or more example implementations of the disclosure;

FIG. 19E is a top view illustrating the one-trunnion asymmetrical patella resurfacing prosthesis with side-plate including one central trunnion, side-plate, with an asymmetrical bearing surface implanted into a prepared left human patella with fixation consisting of multiple screws and one washer according to one or more example implementations of the disclosure;

FIG. 19F is a front view illustrating the one-trunnion asymmetrical patella resurfacing prosthesis with side-plate including one central trunnion, side-plate, with an asymmetrical bearing surface implanted into a prepared left human patella with fixation consisting of multiple screws and one washer according to one or more example implementations of the disclosure;

FIG. 19G is a left view illustrating the one-trunnion asymmetrical patella resurfacing prosthesis with side-plate including one central trunnion, side-plate, with an asymmetrical bearing surface implanted into a prepared left human patella with fixation consisting of multiple screws and one washer according to one or more example implementations of the disclosure;

FIG. 19H is a front view illustrating the one-trunnion asymmetrical patella resurfacing prosthesis with side-plate including one central trunnion, side-plate, with an asymmetrical bearing surface implanted into a prepared left human patella with fixation consisting of multiple screws without a washer according to one or more example implementations of the disclosure;

FIG. 19I is a left view illustrating the one-trunnion asymmetrical patella resurfacing prosthesis with side-plate including one central trunnion, side-plate, with an asymmetrical bearing surface implanted into a prepared left human patella with fixation consisting of multiple screws without a washer according to one or more example implementations of the disclosure;

FIG. 20A is a front view illustrating a metal base of a one-trunnion asymmetrical patella resurfacing prosthesis with side-plate including one central trunnion, side-plate, with an asymmetrical bearing surface according to one or more example implementations of the disclosure;

FIG. 20B is a back view illustrating the metal base of a one-trunnion asymmetrical patella resurfacing prosthesis with side-plate including one central trunnion, side-plate, with an asymmetrical bearing surface according to one or more example implementations of the disclosure;

FIG. 20C is a left view illustrating the metal base of a one-trunnion asymmetrical patella resurfacing prosthesis with side-plate including one central trunnion, side-plate, with an asymmetrical bearing surface according to one or more example implementations of the disclosure;

FIG. 20D is a top view illustrating the metal base of one-trunnion asymmetrical patella resurfacing prosthesis with side-plate including one central trunnion, side-plate, with an asymmetrical bearing surface according to one or more example implementations of the disclosure;

FIG. 20E is a front view illustrating a polyethylene segment of a one-trunnion asymmetrical patella resurfacing prosthesis with side-plate including one central trunnion, side-plate, with an asymmetrical bearing surface according to one or more example implementations of the disclosure;

FIG. 20F is a back view illustrating the polyethylene segment of a one-trunnion asymmetrical patella resurfacing prosthesis with side-plate including one central trunnion, side-plate, with an asymmetrical bearing surface according to one or more example implementations of the disclosure;

FIG. 20G is a left view illustrating the polyethylene segment of a one-trunnion asymmetrical patella resurfacing prosthesis with side-plate including one central trunnion, side-plate, with an asymmetrical bearing surface according to one or more example implementations of the disclosure;

FIG. 20H is a front view illustrating the polyethylene segment of a one-trunnion asymmetrical patella resurfacing prosthesis with side-plate including one central trunnion, side-plate, with an asymmetrical bearing surface according to one or more example implementations of the disclosure;

FIG. 21A is a front view illustrating a flush horizontal two-trunnion asymmetrical patella resurfacing prosthesis with side-plate including two horizontally-arranged trunnions with an asymmetrical bearing surface, and side-plate according to one or more example implementations of the disclosure;

FIG. 21B is a back view illustrating the flush horizontal two-trunnion asymmetrical patella resurfacing prosthesis with side-plate including two horizontally-arranged trunnions with an asymmetrical bearing surface, and side-plate according to one or more example implementations of the disclosure;

FIG. 21C is a left view illustrating the flush horizontal two-trunnion asymmetrical patella resurfacing prosthesis with side-plate including two horizontally-arranged trunnions with an asymmetrical bearing surface, and side-plate according to one or more example implementations of the disclosure;

FIG. 21D is a top view illustrating the flush horizontal two-trunnion asymmetrical patella resurfacing prosthesis with side-plate including two horizontally-arranged trunnions with an asymmetrical bearing surface, and side-plate according to one or more example implementations of the disclosure;

FIG. 21E is a top view illustrating the flush horizontal two-trunnion asymmetrical patella resurfacing prosthesis with side-plate including two horizontally-arranged trunnions with an asymmetrical bearing surface, and side-plate implanted into a prepared left human patella with fixation consisting of multiple screws and washers according to one or more example implementations of the disclosure;

FIG. 21F is a front view illustrating flush horizontal two-trunnion asymmetrical patella resurfacing prosthesis with side-plate including two horizontally-arranged trunnions with an asymmetrical bearing surface, and side-plate implanted into a prepared left human patella with fixation consisting of multiple screws and washers according to one or more example implementations of the disclosure;

FIG. 21G is a left view illustrating the flush horizontal two-trunnion asymmetrical patella resurfacing prosthesis with side-plate including two horizontally-arranged trunnions with an asymmetrical bearing surface, and side-plate implanted into a prepared left human patella with fixation consisting of multiple screws and washers according to one or more example implementations of the disclosure;

FIG. 21H is a front view illustrating the flush horizontal two-trunnion asymmetrical patella resurfacing prosthesis with side-plate including two horizontally-arranged trunnions with an asymmetrical bearing surface, and side-plate implanted into a prepared left human patella with fixation consisting of multiple screws without washers according to one or more example implementations of the disclosure;

FIG. 21I is a left view illustrating flush horizontal two-trunnion asymmetrical patella resurfacing prosthesis with side-plate including two horizontally-arranged trunnions with an asymmetrical bearing surface, and side-plate implanted into a prepared left human patella with fixation consisting of multiple screws without washers according to one or more example implementations of the disclosure;

FIG. 22A is a front view illustrating a flush one-trunnion asymmetrical patella resurfacing prosthesis with side-plate including one central trunnion, side-plate, with an asymmetrical bearing surface according to one or more example implementations of the disclosure;

FIG. 22B is a back view illustrating the flush one-trunnion asymmetrical patella resurfacing prosthesis with side-plate including one central trunnion, side-plate, with an asymmetrical bearing surface according to one or more example implementations of the disclosure;

FIG. 22C is a left view illustrating the flush one-trunnion asymmetrical patella resurfacing prosthesis with side-plate including one central trunnion, side-plate, with an asymmetrical bearing surface according to one or more example implementations of the disclosure;

FIG. 22D is a top view illustrating the flush one-trunnion asymmetrical patella resurfacing prosthesis with side-plate including one central trunnion, side-plate, with an asymmetrical bearing surface according to one or more example implementations of the disclosure;

FIG. 22E is a top view illustrating the flush one-trunnion asymmetrical patella resurfacing prosthesis with side-plate including one central trunnion, side-plate, with an asymmetrical bearing surface implanted into a prepared left human patella with fixation consisting of multiple screws and one washer according to one or more example implementations of the disclosure;

FIG. 22F is a front view illustrating the flush one-trunnion asymmetrical patella resurfacing prosthesis with side-plate including one central trunnion, side-plate, with an asymmetrical bearing surface implanted into a prepared left human patella with fixation consisting of multiple screws and one washer according to one or more example implementations of the disclosure;

FIG. 22G is a left view illustrating the flush one-trunnion asymmetrical patella resurfacing prosthesis with side-plate including one central trunnion, side-plate, with an asymmetrical bearing surface implanted into a prepared left human patella with fixation consisting of multiple screws and one washer according to one or more example implementations of the disclosure;

FIG. 22H is a front view illustrating the flush one-trunnion asymmetrical patella resurfacing prosthesis with side-plate including one central trunnion, side-plate, with an asymmetrical bearing surface implanted into a prepared left human patella with fixation consisting of multiple screws and one washer according to one or more example implementations of the disclosure;

FIG. 22I is a left view illustrating the flush one-trunnion asymmetrical patella resurfacing prosthesis with side-plate including one central trunnion, side-plate, with an asymmetrical bearing surface implanted into a prepared left human patella with fixation consisting of multiple screws and one washer according to one or more example implementations of the disclosure;

FIG. 23A is a front view illustrating a horizontal two-trunnion asymmetrical patella resurfacing prosthesis with integrated side-plate including two horizontally-arranged trunnions with an asymmetrical bearing surface, and an integrated side-plate according to one or more example implementations of the disclosure;

FIG. 23B is a back view illustrating the horizontal two-trunnion asymmetrical patella resurfacing prosthesis with integrated side-plate including two horizontally-arranged trunnions with an asymmetrical bearing surface, and an integrated side-plate according to one or more example implementations of the disclosure;

FIG. 23C is a left view illustrating the horizontal two-trunnion asymmetrical patella resurfacing prosthesis with integrated side-plate including two horizontally-arranged trunnions with an asymmetrical bearing surface, and an integrated side-plate according to one or more example implementations of the disclosure;

FIG. 23D is a top view illustrating the horizontal two-trunnion asymmetrical patella resurfacing prosthesis with integrated side-plate including two horizontally-arranged trunnions with an asymmetrical bearing surface, and an integrated side-plate according to one or more example implementations of the disclosure

FIG. 23E is a top view illustrating the horizontal two-trunnion asymmetrical patella resurfacing prosthesis with integrated side-plate including two horizontally-arranged trunnions with an asymmetrical bearing surface, and an integrated side-plate implanted into a prepared left human patella with fixation consisting of multiple screws and washers according to one or more example implementations of the disclosure;

FIG. 23F is a front view illustrating the horizontal two-trunnion asymmetrical patella resurfacing prosthesis with integrated side-plate including two horizontally-arranged trunnions with an asymmetrical bearing surface, and an integrated side-plate implanted into a prepared left human patella with fixation consisting of multiple screws and washers according to one or more example implementations of the disclosure;

FIG. 23G is a left view illustrating horizontal two-trunnion asymmetrical patella resurfacing prosthesis with integrated side-plate including two horizontally-arranged trunnions with an asymmetrical bearing surface, and an integrated side-plate implanted into a prepared left human patella with fixation consisting of multiple screws and washers according to one or more example implementations of the disclosure;

FIG. 23H is a front view illustrating the horizontal two-trunnion asymmetrical patella resurfacing prosthesis with integrated side-plate including two horizontally-arranged trunnions with an asymmetrical bearing surface, and an integrated side-plate implanted into a prepared left human patella with fixation consisting of multiple screws without washers according to one or more example implementations of the disclosure;

FIG. 23I is a left view illustrating the horizontal two-trunnion asymmetrical patella resurfacing prosthesis with integrated side-plate including two horizontally-arranged trunnions with an asymmetrical bearing surface, and an integrated side-plate implanted into a prepared left human patella with fixation consisting of multiple screws without washers according to one or more example implementations of the disclosure;

FIG. 24A is a front view illustrating a one-trunnion symmetrical patella resurfacing prosthesis with side-plate including one central trunnion, side-plate, with an asymmetrical bearing surface according to one or more example implementations of the disclosure;

FIG. 24B is a back view illustrating the one-trunnion symmetrical patella resurfacing prosthesis with side-plate including one central trunnion, side-plate, with an asymmetrical bearing surface according to one or more example implementations of the disclosure;

FIG. 24C is a left view illustrating the one-trunnion symmetrical patella resurfacing prosthesis with side-plate including one central trunnion, side-plate, with an asymmetrical bearing surface according to one or more example implementations of the disclosure;

FIG. 24D is a top view illustrating the one-trunnion symmetrical patella resurfacing prosthesis with side-plate including one central trunnion, side-plate, with an asymmetrical bearing surface according to one or more example implementations of the disclosure;

FIG. 24E is a top view illustrating the one-trunnion symmetrical patella resurfacing prosthesis with side-plate including one central trunnion, side-plate, with an asymmetrical bearing surface implanted into a prepared left human patella with fixation consisting of multiple screws and one washer according to one or more example implementations of the disclosure;

FIG. 24F is a front view illustrating the one-trunnion symmetrical patella resurfacing prosthesis with side-plate including one central trunnion, side-plate, with an asymmetrical bearing surface implanted into a prepared left human patella with fixation consisting of multiple screws and one washer according to one or more example implementations of the disclosure;

FIG. 24G is a left view illustrating the one-trunnion symmetrical patella resurfacing prosthesis with side-plate including one central trunnion, side-plate, with an asymmetrical bearing surface implanted into a prepared left human patella with fixation consisting of multiple screws and one washer according to one or more example implementations of the disclosure;

FIG. 24H is a front view illustrating the one-trunnion symmetrical patella resurfacing prosthesis with side-plate including one central trunnion, side-plate, with an asymmetrical bearing surface implanted into a prepared left human patella with fixation consisting of multiple screws without a washer according to one or more example implementations of the disclosure; and

FIG. 24I is a left view illustrating the one-trunnion asymmetrical patella resurfacing prosthesis with side-plate including one central trunnion, side-plate, with an asymmetrical bearing surface implanted into a prepared left human patella with fixation consisting of multiple screws without a washer according to one or more example implementations of the disclosure.

Like reference symbols in the various drawings indicate like elements.

### DETAILED DESCRIPTION

Implants may be used, e.g., when one or more human (or animal) body parts need replacing. For example, known patella resurfacing implants may have been developed as part of total knee arthroplasty and patellofemoral arthroplasty systems, in order to treat such problems as, e.g., osteoarthritis of the knee and specifically the patellofemoral joint. Such implants may be associated with, e.g., bone thermal necrosis, which may adversely affect the patella bone strength over time. For instance, known implants may rely upon usage of bone cement in order to achieve stabile fixation of the patella component to the surgically prepared and shaped patella. While cement may be effective for surgical fixation of the patella prosthesis to the patella itself, the cement is typically applied at temperatures well above normal body temperature. In some situations, the increased temperature profile associated with cemented prosthesis implantation may be associated with bone thermal necrosis, which may adversely affect the patella bone strength over time.

As will be discussed in greater detail below, the example patella resurfacing prosthesis may, in some implementations, be able to lessen or entirely avoid thermal necrosis by, e.g., employing screw fixation rather than cement application for achieving implant fixation. In some implementations, uncemented implantation may help reduce the risk of aseptic loosening following implantation. In some implementations, a porous surface along the undersurface of the patella resurfacing prosthesis may allow for additional implant stability through osseous-integration over time.

In some implementations, as will also be discussed in greater detail below, the example orthopedic implants may relate generally to patellofemoral arthroplasty and to a patella resurfacing prosthesis, which may be fitted to a shaped patella and implanted with or without use of bone cement. The patella resurfacing prosthesis may be fixed into the residual native bone of the patella using, e.g., screw fixation. In some implementations, implant and fixation options may allow adaptation of fixation based on, e.g., underlying bone quality as factors including osteoporosis may affect fixation stability. Compromised patella bone quality may increase peri-prosthetic fracture risk. In some implementations, the patella resurfacing prosthesis may offer increased construct strength using additional plates and screws when peri-prosthetic fracture risk is of concern. In some implementations, these scalable options may allow for individuated surgical decision making based on localized bone quality.

In some implementations, as will also be discussed in greater detail below, the example patella resurfacing prosthesis may include one or more threaded screw holes to accommodate anterior screw fixation. In certain implementations, the capability for use of anterior screw fixation for the patella component may not require cement application, which may help avoid the potential for cement-associated thermal necrosis. In some implementations, fixation may be adjusted on an individualized basis through, e.g., the selected implementation of the patella resurfacing prosthesis, screw fixation, etc., where construct stability may be increased based on, e.g., pre-surgical planning. In some implementations, implant selection with the example patella resurfacing prosthesis may take into account fracture risk and may allow the surgeon to add supplemental plates as needed, which is a feature not necessarily available with known patella resurfacing prosthesis. That is, known patella resurfacing prosthesis may lack a patella component that accommodates attachment of screw and plate fixation.

In some implementations, unlike known patella resurfacing prosthesis, the example patella resurfacing prosthesis of the present disclosure may enable further compression along the prosthesis-bone interface (e.g., using anterior screw fixation), where osseous-integration over time along some or all of the porous undersurface of the patella component may be allowed. In certain implementations, cement may not be needed to be applied along the prosthesis-bone interface, and bone may then grow into a porous prosthesis undersurface. In some implementations, this may allow for increasing construct stability subsequent to the post-operative period. In certain implementations, the patella resurfacing prosthesis may be implanted along with an existing femoral trochlear component in order to achieve complete patellofemoral resurfacing arthroplasty, including both the patellar and femoral trochlear articular surfaces.

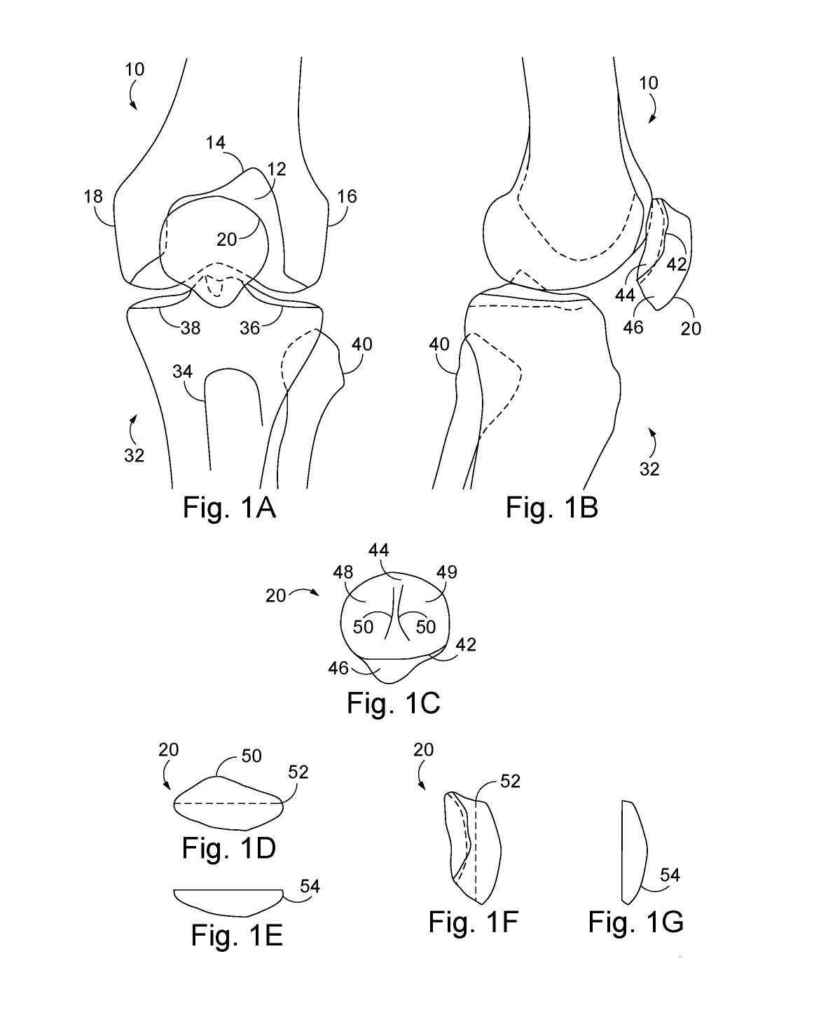

Referring at least to example FIG. 1A, a front view from anterior of a human left knee is shown illustrating multiple osseous structures. The distal femur 10 includes a trochlear surface 12, trochlear border 14, lateral femoral condyle 16, and medial femoral condyle 18. The patella 20 articulates with the trochlear surface. The proximal tibia 32 contains a lateral tibial plateau 36, medial tibial plateau 38, and tibial tuberosity 34. A proximal fibula 40 is also visible. Referring at least to example FIG. 1B, there is shown a left view further illustrating a human left knee. The patella 20, includes an articular border 42, articular surface 44, and extraarticular segment 46. Referring at least to example FIG. 1C, there is shown a back view of a human left patella further illustrating the articular border 42, articular surface 44, and extraarticular segment 46. Along the articular surface is the lateral facet 48 and medial facet 49, both separated by the patella ridge 50. Referring at least to example FIG. 1D, there is shown a top view of a human left patella with an osteotomy reference line 52 for planning implantation of patella resurfacing prosthesis. This osteotomy reference line approximates the middle of the patella between the most anterior and most posterior surfaces of the patella as visualized in this view. Referring at least to example FIG. 1E, there is shown a top view of the residual patella 54, which is the anterior aspect of the patella retained following a vertically-oriented bone cut for a patella resurfacing prosthesis according to one or more implementations of the present disclosure. Referring at least to example FIG. 1F, there is shown a left view of a human left patella further illustrating the osteotomy reference line 52 for planning implantation of the patella resurfacing prosthesis. The reference line also approximates the middle of the patella between the most anterior and most posterior surface of the patella in this view. Referring at least to example FIG. 1G, there is shown a left view of the residual patella 54, which is the anterior aspect of the patella retained following a vertically-oriented osteotomy for implantation of a patella resurfacing prosthesis according to one or more implementations of the present disclosure.

As used herein, the term patella resurfacing prosthesis, patella resurfacing implant, and patella component, may be used interchangeably.

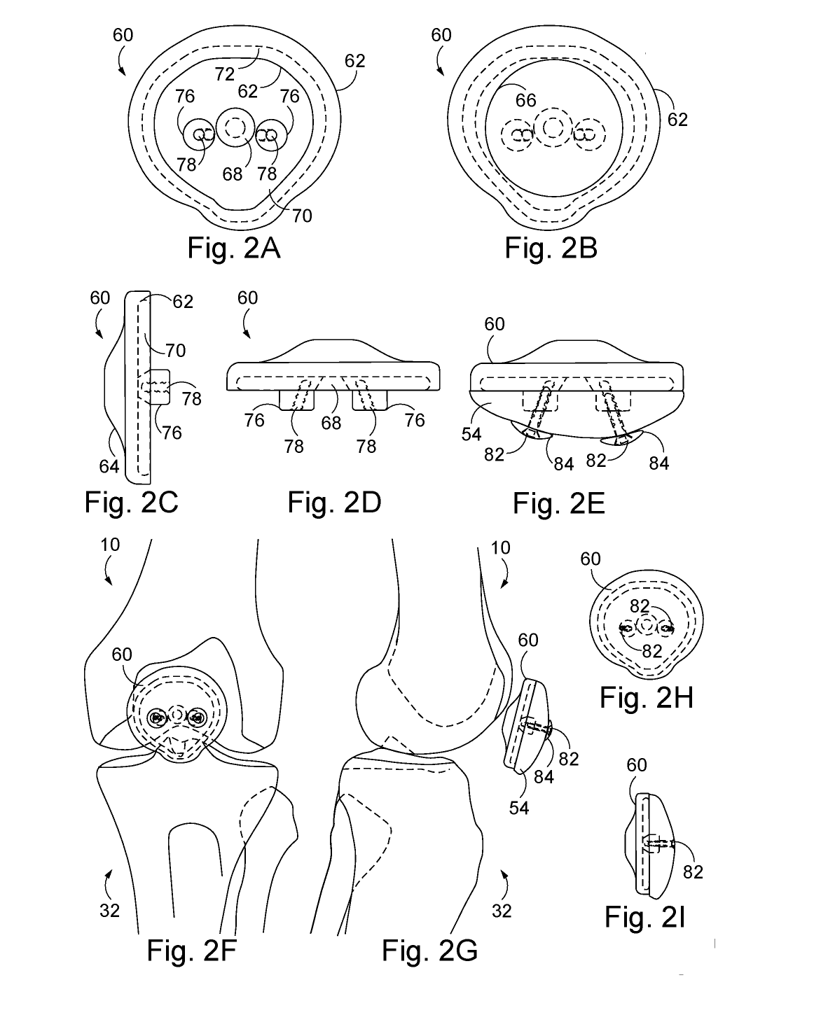

As discussed above and referring also at least to the example implementations of FIGS. 2A-24I, a patella resurfacing prosthesis may include but is not limited to a patella resurfacing implant. In some implementations, the patella resurfacing prosthesis may include one or more threaded holes along a front aspect of the patella resurfacing implant, wherein the one or more threaded holes along the front aspect of the patella resurfacing implant may be configured to fixate the patella resurfacing implant to a patella of a user. In some implementations, the patella resurfacing prosthesis may include one or more trunnions, wherein the one or more threaded holes along the front aspect of the patella resurfacing implant may be within the one or more trunnions respectively. In some implementations, the one or more trunnions may include two or more trunnions arranged horizontally. In some implementations, the patella resurfacing prosthesis may include a polyethylene segment. For example, and referring at least to the example implementation of FIG. 2A, a front view is shown illustrating various example aspects of a horizontal two-trunnion symmetrical patella component 60, in accordance with one or more example implementations of the present disclosure. In some implementations, the outer surface in the present implementation may consist of polyethylene as illustrated with its outer and inner polyethylene periphery 62. In certain implementations, the polyethylene may extend through the base (e.g., a metal base) in a cone form creating flush polyethylene retainer 68, with the widest aspect of the cone situated along the front aspect of the metal base. Some implementations may include metal base 70, which may be situated along the front of the polyethylene segment. The metal base may extend below the surface of the polyethylene as illustrated by the recessed edge of metal base 72. The metal base may include two or more of trunnion 76, horizontally-arranged and joined along the front of metal base 70. The trunnions may extend the front of the metal base and may include threaded hole 78 for accommodation of screws. In some implementations, the threaded hole(s) may be angled in an inward fashion.

Example FIG. 2B shows a back view further illustrating the prosthesis in one or more implementations. The back of the prosthesis may include a dome-shaped, symmetrical bearing surface, with the polyethylene raised segment border 66 demarcating the periphery of the dome. Example FIG. 2C shows a left view of the prosthesis including its bearing surface 64. Metal base 70 may be further visualized along with trunnion 76 and threaded hole 78. Example FIG. 2D shows a top view further demonstrating the trunnions with threaded hole 78 present in each, and the flush polyethylene retainer 68. The orientation of the two trunnions may be further illustrated along with the orientation of the threaded holes which may be angled inward, toward the each other, from front to the back. The polyethylene retainer may help to keep the metal base joined to the polyethylene. The metal base may extend further into the polyethylene below the front surface of the polyethylene within the recessed edge of metal base 72 to further help retain the polyethylene within the metal base. Example FIG. 2E shows a top view illustrating the prosthesis implanted into a left human residual patella. In some implementations, the one or more trunnions may be configured to receive one or more screws with one or more washers. For instance, in some implementations, the implant may be secured with screw fixation, with screw 82 inserted through washer 84 and threaded and secured to the threaded hole of one or more of the respective trunnions.

Example FIGS. 2F and 2G show front and left views, respectively, further illustrating the example implanted and secured prosthesis according to one or more implementations. Example FIGS. 2H and 2I show front and left views illustrating the same prosthesis secured with screw fixation but without use of supplemental washers. In some implementations, the patella prosthesis may either be secured using screws, which may be implanted with the screw heads flush to the patella anterior cortical bone as illustrated, or additionally/alternately, may be secured with the screw heads above of the anterior cortex. The patella resurfacing prosthesis may have both a left knee and a right knee version. Although a left patella resurfacing prosthesis version is illustrated in figures, the right-sided version of the patella resurfacing prosthesis may be a mirror image of the left-sided version. This mirror image may be applied to the back of the prosthesis, articular bearing surface, and to the front, along which the threaded holes and trunnions may be situated.

In some implementations, the patella resurfacing prosthesis may be implanted through known surgical techniques, and with some slight modifications. The patient may be positioned supine with the operative knee placed in extension to release tension from the extensor mechanism of the knee. A lateral parapatellar incision may be performed along the anterior aspect of the operative patella, from the tibial tuberosity to approximately, e.g., 2 centimeters proximal to the patella, and may be carried through the underlying subcutaneous tissue. A lateral parapatellar arthrotomy may then performed. Inversion of the patella may be performed through this surgical exposure allowing for optimal surgical exposure during the procedure of the articular surface and peripheral patella from its lateral aspect. The skin may then be reflected further from the retinacular tissue overlying the patella, in order to visualize the anterior aspect of the patella. This may allow for placement secondary retraction of the skin during guide wire placement and screw hole drilling and placement of screws, washers and other fixation devices as required. A ridge of bone may then be exposed along the periphery of the patella, as needed for the patella osteotomy, which may be performed as in FIGS. 1D to 1G and osteotomy reference line 52. The osteotomy may be performed in standard fashion and surgical instrumentation as known in patellofemoral resurfacing. The subcutaneous tissue may be protected and retracted while the patella is stabilized with surgical clamps, and an oscillating surgical bone saw is utilized for performing the osteotomy. After completion of the osteotomy and freeing and removal of the posterior aspect of the patella, which may include the articular surface, two holes may be drilled partially through the patella for accommodation of the two trunnions in this example implementation, using a surgical caliper (or other suitable instrument) for reference and guide wires, for screw fixation. The same drill guides may then be used for completion of the narrower holes required for secondary anterior placement of the two screws for fixation of the prosthesis. The holes drilled for the trunnions may align with the prosthesis as final implantation conforms to a shape-fit. Once the osteotomy is completed along with drilling of the trunnion holes and screw holes the prosthesis is fitted and secured through insertion of the screws from, e.g., the anterior aspect, with or without washers, and symmetrical tightening of each screw.

In some implementations, the horizontal orientation of the two trunnions may provide for additional construct stability against tensile forces acting along the medial and lateral aspects of the patella and component interface. The subcutaneous tissue, retinacular tissue and skin incision may be successively closed with suture fixation and standard postoperative protocols for patellofemoral resurfacing arthroplasty may be initiated. The patella resurfacing prosthesis may be implanted along with a femoral trochlear component to achieve complete patellofemoral resurfacing arthroplasty, which may include the femoral articular surface. Additionally/alternately, a medially based dissection may be performed instead of a lateral dissection by, e.g., performing the surgical incision through a medial parapatellar approach along with a medial arthrotomy.

In some implementations, the patella resurfacing prosthesis may include a base, wherein the one or more threaded holes along the front aspect of the patella resurfacing implant may be within the base. In some implementations, the base may include a metal base. For example, the patella resurfacing prosthesis in one or more example implementations may be manufactured using a polyethylene articular surface and a metal base. The polyethylene may include standard polyethylene or highly-crosslinked polyethylene. In some implementations, the metal base, and the screws, may consist of high carbon cobalt chrome (CoCr) alloy. The metal base, and the screws, may be made of an alternate metal or metal alloy including, e.g., stainless steel, titanium, or another suitable material. The metal base, in some implementations, may be formed as cast, forged, milled or drilled through machining processes or a combination of methods. Standard machining techniques may be used for creating or tapping the threaded holes. The exposed front of the metal base, uncovered by the polyethylene segment, may have a surface to induce osseous-integration along the implant-bone interface. This surface may consist of a porous, textured, granular and/or beaded surface coating. The polyethylene segment and metal base may be manufactured by, e.g., forming the polyethylene segment as cast around the metal base. The polyethylene segment may be injection molded around the completed metal base, with the metal base manufactured in advance of the polyethylene segment. Additionally/alternately, the polyethylene segment may be produced in two pieces with the plastic, then joined around the completed metal base through plastic welding, further smoothed during production, and standard manufacturing techniques. The bearing surface may be highly polished during the manufacturing process.

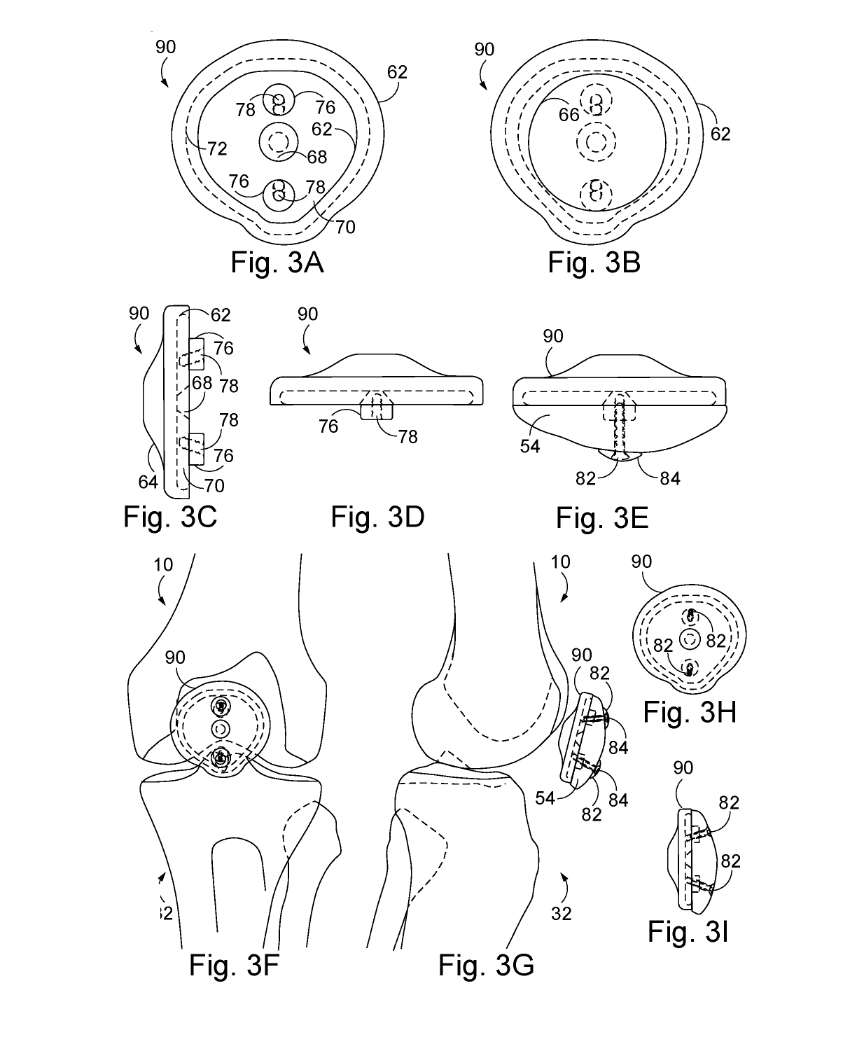

In some implementations, the one or more trunnions may include two or more trunnions arranged vertically. For example, and referring at least to the example implementation of FIG. 3A, there is shown a front view illustrating various example aspects of a vertical two-trunnion symmetrical patella component 90. The prosthesis and steps for implantation may be the same or similar as that illustrated and described in FIGS. 2A to 2I with a few example differences, which are described below. In some implementations, trunnion 78 positioning may be vertically-arranged along the front of the prosthesis. Example FIG. 3B shows a back view illustrating the polyethylene symmetrical bearing surface with its polyethylene raised segment border 66. Example FIGS. 3C and 3D show left and top views, respectively, illustrating the two vertically-arranged trunnions and a symmetrical bearing surface. Example FIGS. 3E, 3F and 3G show top, front and left views, respectively, illustrating the prosthesis implanted into a left human residual patella 54 with screw 82 and washer 84 secured to the threaded holes of both trunnions. Example FIGS. 3H and 3I show front and left views, respectively, illustrating the prosthesis implanted into a left human patella with two screws without use of washers. The steps for implantation may be the same or similar as in FIGS. 2A to 2I, except the holes drilled for the trunnions and screws may be in a vertical pattern in order to achieve a shape-fit. In some implementations, the vertical two-trunnion arrangement may provide additional construct stability against bending forces tensile forces acting along the superior and inferior aspects of the patella and component interface.

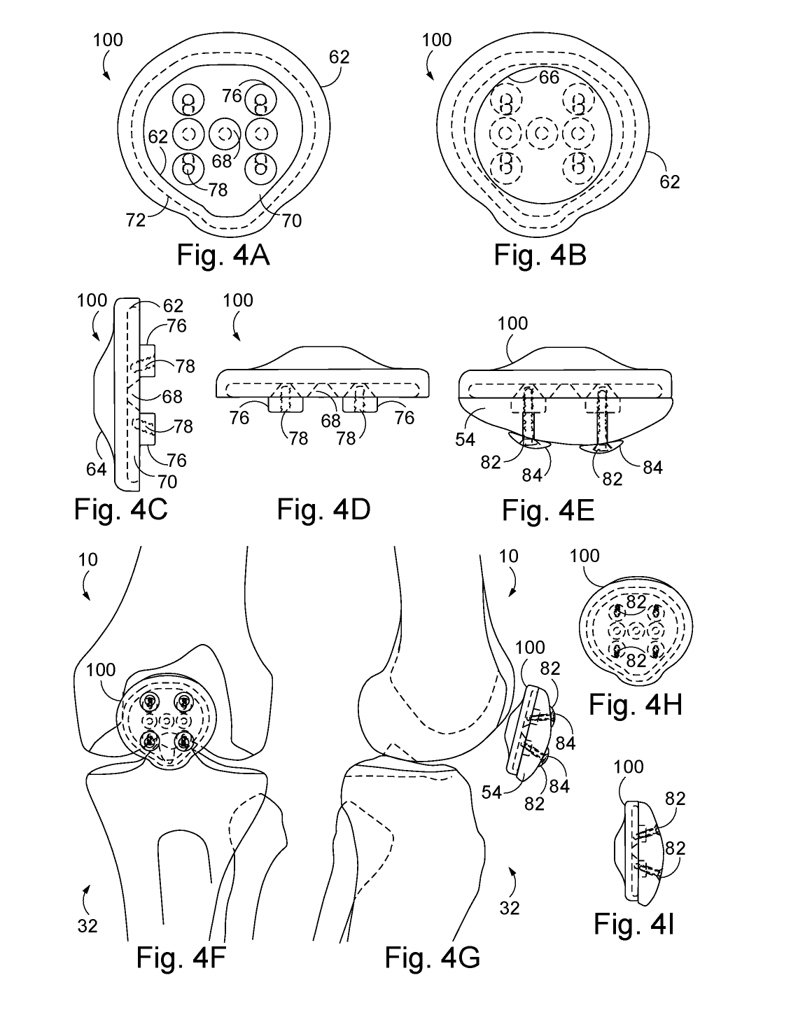

In some implementations, and referring at least to the example implementation of FIG. 4A, there is shown a front view illustrating various example aspects of a four-trunnion symmetrical patella component 100. In the example, the prosthesis and steps for implantation may be the same or similar as that illustrated and described in FIGS. 2A to 2I with a few example differences, which are described below. Trunnion 78 positioning in this example implementation is both vertically and horizontally oriented along the front of the prosthesis, as four trunnions may be joined to metal base 70. In some implementations, there may be included one or more (e.g., three) flush polyethylene retainer 68 extensions. Example FIG. 4B shows a back view illustrating the polyethylene symmetrical bearing surface with its polyethylene raised segment border 66. Example FIGS. 4C and 4D show left and top views, respectively, illustrating the orientation of the trunnions and a symmetrical bearing surface. Example FIGS. 4E, 4F and 4G show top, front and left views, respectively, illustrating the prosthesis implanted into a left human residual patella with four of screw 82 and washer 84 secured to the threaded holes of one or more of the four trunnions.

In some implementations, the one or more trunnions may be configured to receive one or more screws without one or more washers. For example, and referring at least to the example implementations of FIGS. 4H and 4I, there is shown front and left views, respectively, illustrating the prosthesis implanted into a left human residual patella with one or more (e.g., two) screws without use of washers. The steps for implantation may be the same as in FIGS. 2A to 2I, except four holes may be drilled for the trunnions matching the example configuration in order to achieve a shape-fit. In some implementations, the inclusion of the four-trunnions arrangement may provide increased construct stability and increased resistance to tensile forces along the patella and component interface.

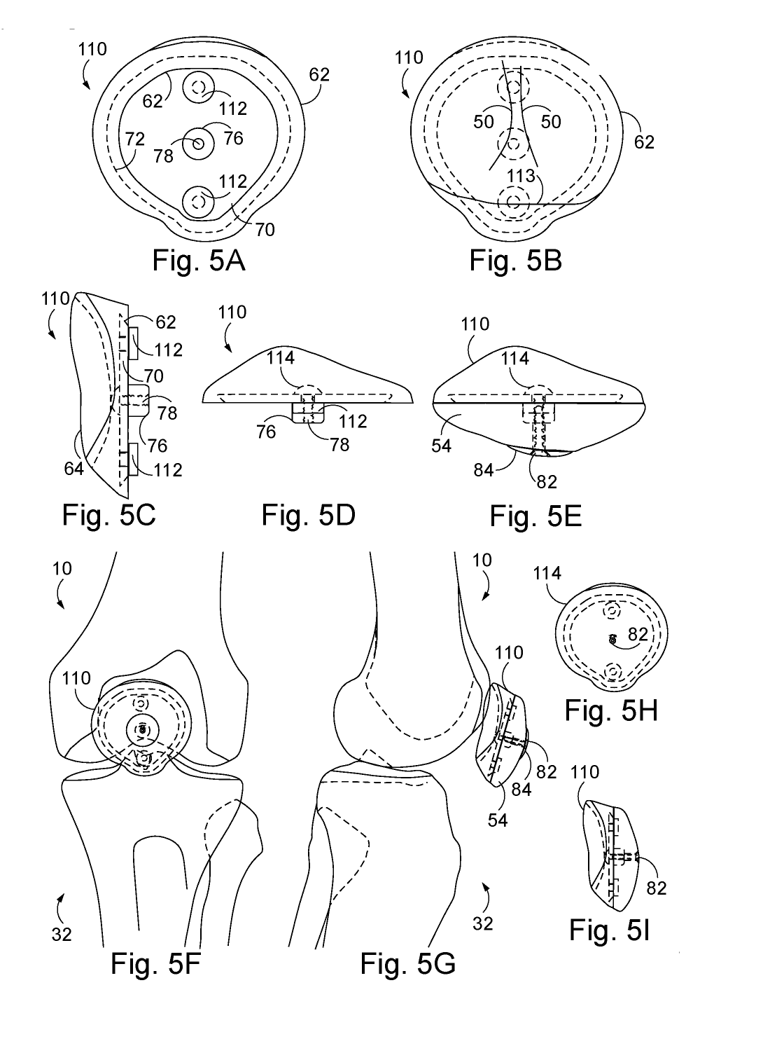

It will be appreciated that some implementations may include one trunnion, or more than one trunnion. In some implementations, at least one trunnion of the one or more trunnions may be a central trunnion. For example, and referring at least to the example implementation of FIG. 5A, there is shown a front view illustrating various example aspects of a one-trunnion asymmetrical patella component 110. The prosthesis and steps for implantation may be the same or similar as that illustrated and described in FIGS. 2A to 2I with a few example and non-limiting differences, which are described below. In the example, the positioning of trunnion 78 may be centrally-oriented along the front of the prosthesis, with one trunnion joined to metal base 70. The polyethylene may extend through the metal base to form round prominences, which may widen in front of the metal base forming two cylindrical, non-flush polyethylene retainer 112 extensions. These may be included to, e.g., provide additional rotational stability when the prosthesis is implanted into the patella, as well as to further resistance to separation of the metal base from the polyethylene.

In some implementations, the one or more trunnions may be arranged with an asymmetrical bearing surface. Furthermore, in some implementations, and referring at least to the example implementation of FIG. 5B, the asymmetrical bearing surface may be shaped to mimic the contours and anatomical bearing surface of the native human patella, including the contours of the medial facet, lateral facet and patella ridge. Inclusion of a bearing surface designed to mimic the native anatomical patella may help to preserve more natural patellofemoral joint kinematics. For instance, and referring to the example implementation of FIG. 5B, there is shown a back view illustrating the polyethylene asymmetrical bearing surface with an articular border 113, similar in shape to the anatomical patella articular surface. Also similar to the native anatomy of the human residual patella is patella ridge 50 along the bearing surface. FIGS. 5C and 5D show left and top views, respectively, illustrating the orientation of the central trunnion and asymmetrical bearing surface. The bearing surface may simulate the shape of the native patella anatomy. The polyethylene may include screw void 114 to safely accommodate screws when they are tightened, e.g., just beyond the metal base. Example FIGS. 5E, 5F and 5G show top, front and left views, respectively, illustrating the prosthesis implanted into a left human residual patella with screw 82 and washer 84 secured to the threaded hole of the centrally-oriented trunnion. Example FIGS. 5H and 5I show front and left views, respectively, illustrating the prosthesis implanted into a left human residual patella with one screw without use of a washer. The steps for implantation may be the same or similar as in FIGS. 2A to 2I except one hole is drilled for the central trunnion matching the configuration of an example implementation in order to achieve a shape-fit. Two holes may be drilled, above and below the hole for the central trunnion for both of the non-flush polyethylene retainer 112 prominences, and partially into the patella to a depth matching their height. In some implementations, inclusion of one trunnion may reduce requisite screw placement. In some implementations, the two non-flush polyethylene retainer prominences may provide rotational stability upon implantation to further resist rotational forces along the patella-prosthesis interface without placement of additional screws. In some implementations, the asymmetrical bearing surface may simulate the human residual patella bearing surface and may be implanted into combination with an anatomical femoral trochlear component to achieve joint kinematics more closely mimicking the native knee joint than a dome shaped bearing surface.

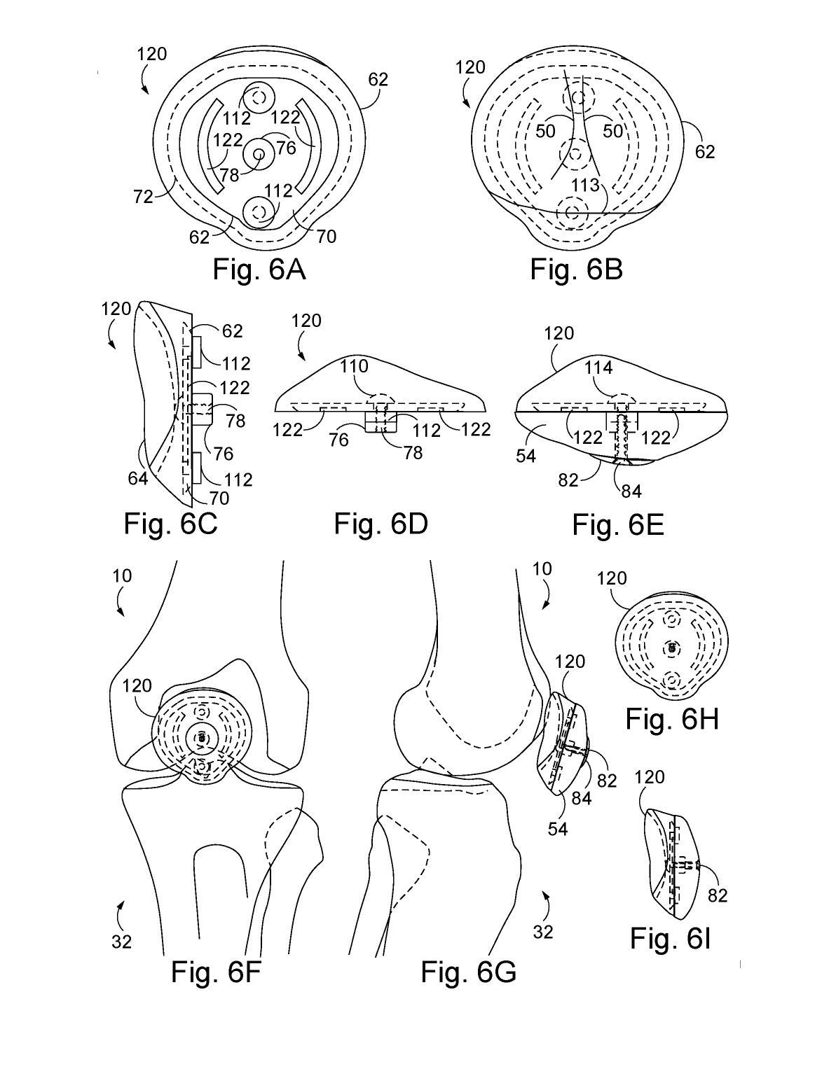

In some implementations, the patella resurfacing prosthesis may include one or more channels configured to receive bone cement. For instance, and referring to the example implementation of FIG. 6A, there is shown a front view illustrating various example aspects of an example implementation of a one-trunnion channeled asymmetrical patella component 120. The prosthesis and steps for implantation may be the same or similar as that illustrated and described in FIGS. 5A to 5I with a few example and non-limiting differences, which are described below. The front of the metal base may include channel 122 in duplicate. This channel may be added in order to provide for further bone cement mantle thickness when implanted into a cemented fashion. This channel may allow both excess bone cement to enter the included channels and also to form two ridges of thicker cement mantles within the channels to provide further rotational resistance. Example FIG. 6B shows a back view illustrating the polyethylene asymmetrical bearing surface with articular border 113, similar in shape to the anatomical patella bearing surface and articular border. Example FIGS. 6C and 6D show left and top views, respectively, illustrating the orientation of the central trunnion, asymmetrical bearing surface and channels. Example FIGS. 6E, 6F and 6G show top, front and left views, respectively, illustrating the prosthesis implanted into a left human residual patella with screw 82 and washer 84 secured to the threaded holes of the centrally-oriented trunnion. Example FIGS. 6H and 6I show front and left views, respectively, illustrating the prosthesis implanted into a left human residual patella with one screw without use of a washer. The steps for implantation may be the same or similar as in FIGS. 5A to 5I except cement may be applied to the osteotomized surface of the patella after the patella bone it is both osteotomized and requisite holes drilled. In some implementations, the inclusion channels may accommodate cemented application when bone quality within the native patella is diminished and additional fixation is desired in addition to screw fixation.

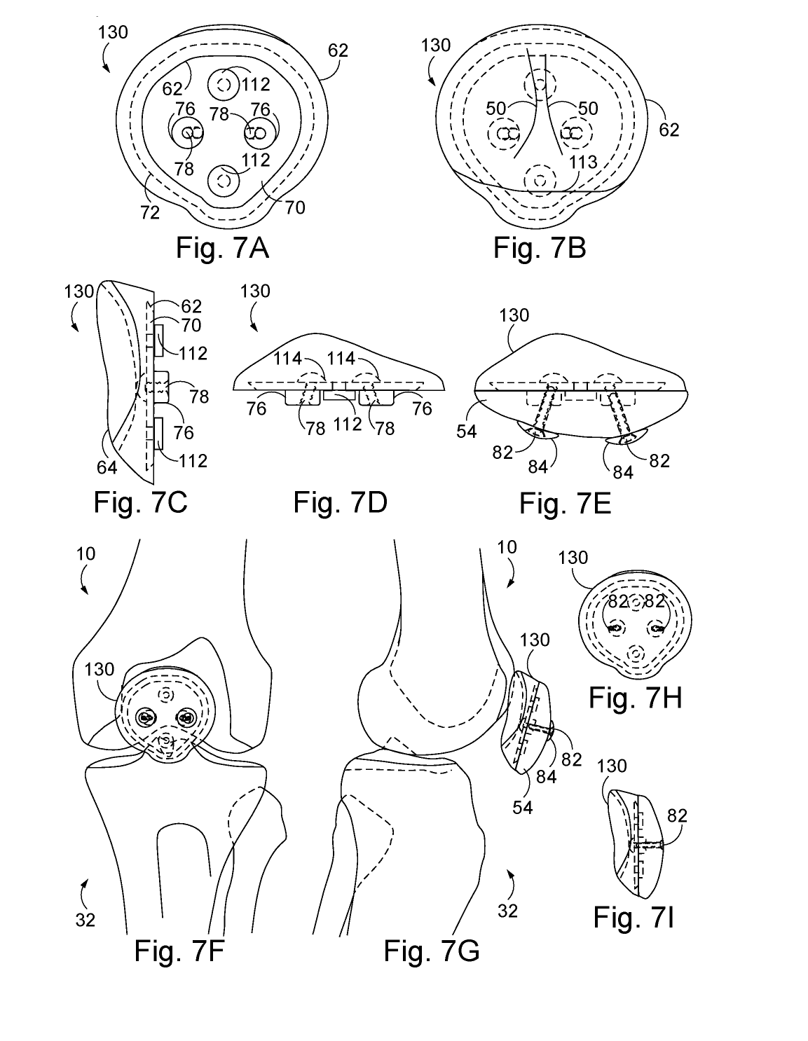

In some implementations, and referring at least to the example implementation of FIG. 7A, there is shown a front view illustrating various example aspects of a horizontal two-trunnion asymmetrical patella component 130. The prosthesis and steps for implantation may be the same or similar as that illustrated and described in FIGS. 5A to 5I with a few example and non-limiting differences, which are described below. The front of the metal base may include two horizontally-arranged trunnions. Example FIG. 7B is a back view illustrating the polyethylene asymmetrical bearing surface with articular border 113, similar in shape to the anatomical patella bearing surface and articular border. Example FIGS. 7C and 7D are left and top views, respectively, illustrating the orientation of the two trunnions, asymmetrical bearing surface and threaded holes angled inward, toward the each other, from front to the back. In some implementations, the polyethylene may include two of screw void 114 to safely accommodate two screws when they are tightened just beyond the metal base. Example FIGS. 7E, 7F and 7G are top, front and left views, respectively, illustrating the prosthesis implanted into a left human residual patella using two of screw 82 and washer 84 secured to the threaded holes of both trunnions. Example FIGS. 7H and 7I are front and left views, respectively, illustrating the prosthesis implanted into a left human residual patella with two screws without use of washers. The steps for implantation may be the same or similar as in FIGS. 5A to 5I, except two horizontally-arranged holes may be drilled partially through the patella to accommodate the two trunnions as well as the two holes for the anteriorly placed screws for each associated threaded hole 78. In some implementations, the combination of an anatomical bearing surface simulating the native patella and inclusion of a horizontal two-trunnion arrangement may provide for additional construct stability against tensile forces acting along the medial and lateral aspects of the patella and component interface.

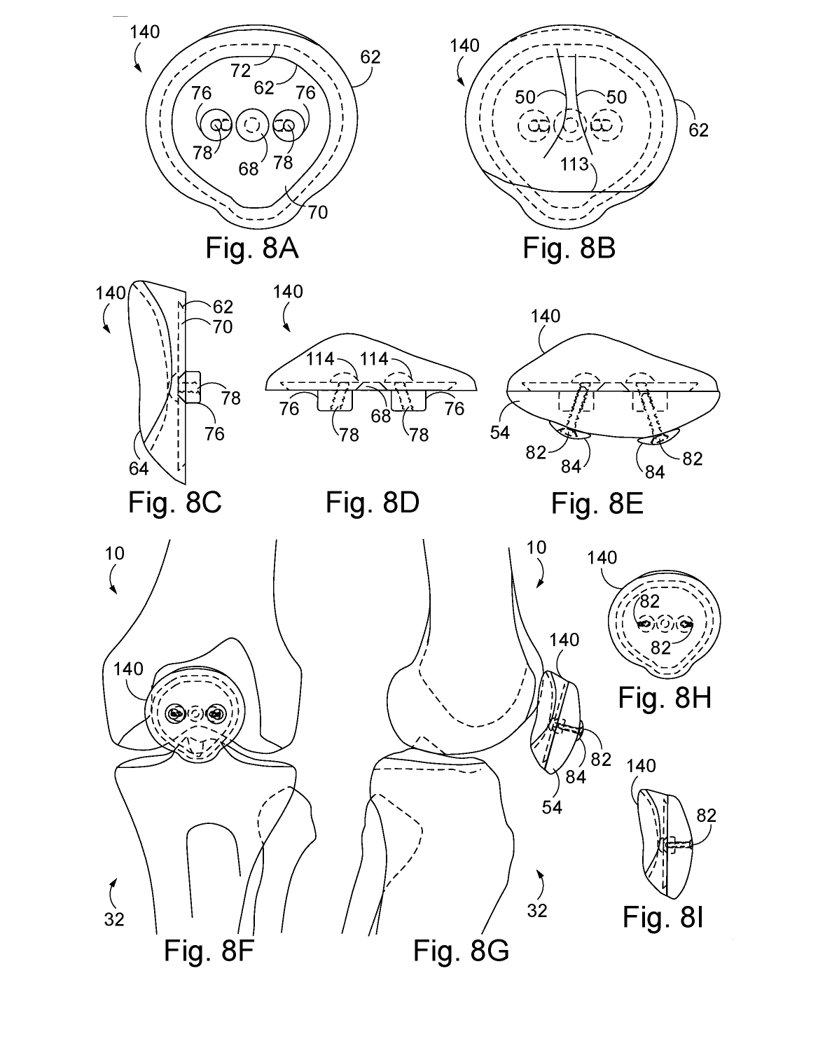

In some implementations, and referring at least to the example implementation of FIG. 8A, there is shown a front view illustrating various example aspects of a flush horizontal two-trunnion asymmetrical patella component 140. The prosthesis and steps for implantation may be the same or similar as that illustrated and described in FIGS. 7A to 7I with a few example and non-limiting differences, which are described below. The front of the metal base may include a flush polyethylene retainer 68 extension so the polyethylene does not extend beyond the metal base of the prosthesis. Example FIG. 8B is a back view illustrating the polyethylene asymmetrical bearing surface with articular border 113, similar in shape to the anatomical patella bearing surface and articular border. Example FIGS. 8C and 8D are left and top views, respectively, illustrating the orientation of the two trunnions, asymmetrical bearing surface and threaded holes angled toward the each other, from front to the back. Example FIGS. 8E, 8F and 8G are top, front and left views, respectively, illustrating the prosthesis implanted into a left human residual patella with using two of screw 82 and washer 84 threaded and secured to the threaded holes of both trunnions. Example FIGS. 8H and 8I are front and left views, respectively, illustrating the prosthesis implanted into a left human residual patella with two screws without use of washers. The steps for implantation may be the same or similar as in FIGS. 8A to 8I except that no additional drill holes are made above and below the trunnions as the polyethylene is flush to the metal base in this implementation and does not require additional drill holes. In some implementations, the combination of inclusion an anatomical bearing surface, horizontal two-trunnion arrangement for controlling tensile forces acting along the medial and lateral aspects of the patella and component interface, and flush polyethylene retainer 68 extensions may avoid secondary drilling for accommodation of non-flush polyethylene retainer prominences.

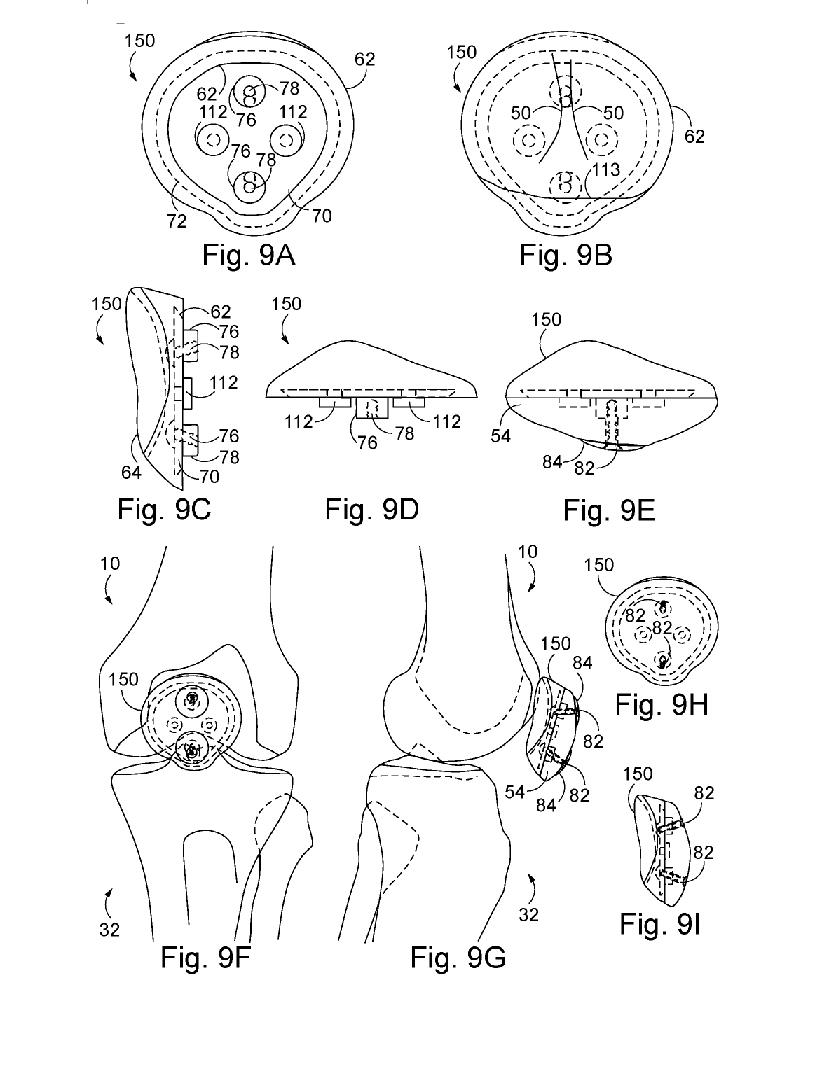

In some implementations, and referring at least to the example implementation of FIG. 9A, there is shown a front view illustrating various example aspects of a vertical two-trunnion asymmetrical patella component 150. The prosthesis and steps for implantation may be the same or similar as that illustrated and described in FIGS. 7A to 7I with a few example and non-limiting differences, which are described below. The front of the metal base may include two non-flush polyethylene retainer 112 prominences providing additional rotational stability when implanted. Example FIG. 9B is a back view illustrating the polyethylene asymmetrical bearing surface with an articular border 113, similar in shape to the anatomical patella bearing surface and articular border. Example FIGS. 9C and 9D are left and top views, respectively, illustrating the orientation of the two trunnions, asymmetrical bearing surface and threaded holes angled toward the each other, from front to the back. Example FIGS. 9E, 9F and 9G are top, front and left views, respectively, illustrating the prosthesis implanted into a left human residual patella with using two of screw 82 and washer 84 secured to the threaded holes of both trunnions. Example FIGS. 9H and 9I are front and left views, respectively, illustrating the prosthesis implanted into a left human residual patella with two screws without use of washers. The steps for implantation may be the same or similar as in FIGS. 7A to 7I except that the non-flush polyethylene retainer 112 prominences may be along the left and right aspects along the front of the prosthesis and the trunnions may be along the top and bottom. In some implementations, screw holes may be drilled along superior and inferior aspects of the patella. Holes may be drilled for accommodation of the non-flush polyethylene retainers. In some implementations, the combination of inclusion of an anatomical bearing surface with implant stability afforded through the vertical two-trunnion arrangement may help with resistance to tensile forces acting along the superior and inferior aspects of the patella and component interface.

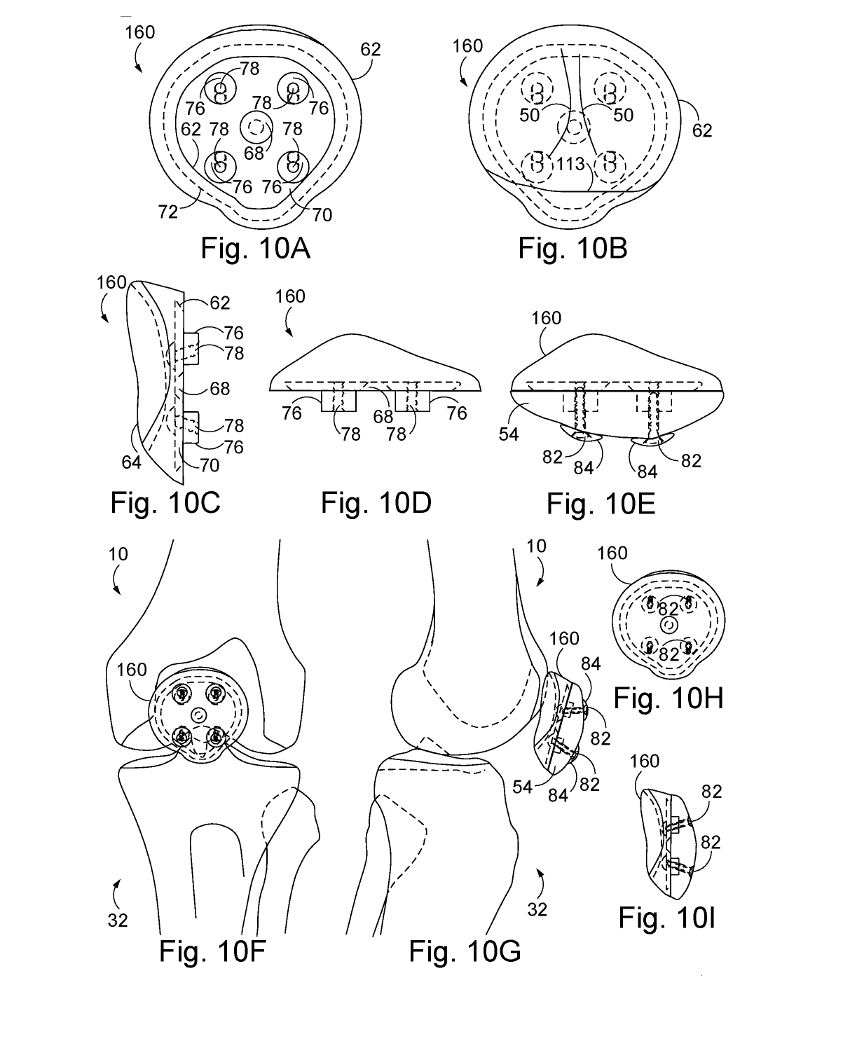

In some implementations, and referring at least to the example implementation of FIG. 10A, there is shown a front view illustrating various example aspects of a four-trunnion asymmetrical patella component 160. The prosthesis and steps for implantation may be the same or similar as that illustrated and described in example FIGS. 8A to 8I with a few example and non-limiting differences, which are described below. The front of the metal base may include a flush polyethylene retainer 68. Example FIG. 10B is a back view illustrating the polyethylene asymmetrical bearing surface with an articular border 113, similar in shape to the anatomical patella bearing surface and articular border. Example FIGS. 10C and 10D are left and top views, respectively, illustrating the orientation of the four trunnions, asymmetrical bearing surface and angled threaded holes. Example FIGS. 10E, 10F. and 10G are top, front and left views, respectively, illustrating the prosthesis implanted into a left human residual patella using four of screw 82 and washer 84 secured to the threaded holes of one or more of the four trunnions. Example FIGS. 10H and 10I are front and left views, respectively, illustrating the prosthesis implanted into a left human residual patella with four screws without use of washers. The steps for implantation may be the same or similar as in example FIGS. 8A to 8I but with four trunnions. Four trunnion and screw holes may therefore be drilled in the osteotomized the patella. In some implementations, the combination of an anatomical bearing surface with implant stability afforded by the four-trunnion arrangement may enable increased implant stability and resistance to tensile forces along the patella and component interface.

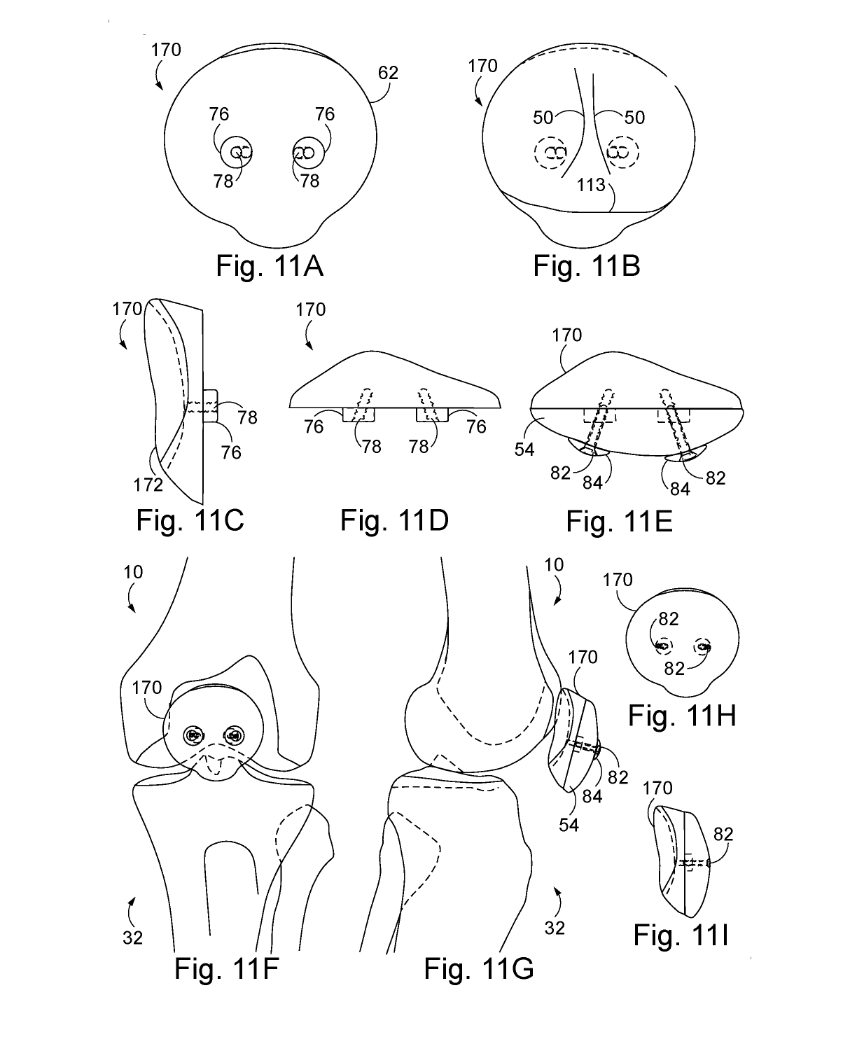

In some implementations, and referring at least to the example implementation of FIG. 11A, there is shown a front view illustrating various example aspects of a metal horizontal two-trunnion asymmetrical patella component 170. The prosthesis and steps for implantation may be the same or similar as that illustrated and described in example FIGS. 8A to 8I with a few example and non-limiting differences, which are described below. The prosthesis may be partly or fully metal and without a polyethylene segment. Example FIG. 11B is a back view illustrating the asymmetrical bearing surface with articular border 113, similar in shape to the anatomical patella bearing surface and articular border. The prosthesis may be fully metal, including the articular bearing surface. Example FIGS. 11C and 11D are left and top views, respectively, illustrating trunnion 76 arrangement and each threaded hole 78, and asymmetrical bearing surface. Example FIGS. 11E, 11F and 11G are top, front and left views, respectively, illustrating the prosthesis implanted into a left human residual patella with two of screw 82 and washer 84 secured to the threaded holes of both trunnions. Example FIGS. 11H and 11I are front and left views, respectively, illustrating the prosthesis implanted into a left human residual patella with two screws without use of washers. The steps for implantation may be the same or similar as in example FIGS. 8A to 8I. In some implementations, since the prosthesis may be fully metal, it may be made of one material reducing manufacturing steps and also may provide greater mechanical strength properties as compared to a metal and polyethylene, or a fully polyethylene prosthesis. The horizontal two-trunnion arrangement may help enable resistance to tensile forces along the medial and lateral aspects of the patella and component interface.

In some implementations, the patella resurfacing prosthesis may be manufactured fully with metal. The metal may include, e.g., a high carbon cobalt chrome (CoCr) alloy. In some implementations, the patella resurfacing prosthesis may also be made of an alternate metal or metal alloy including, e.g., stainless steel, titanium, or another suitable material. The patella resurfacing prosthesis may be formed as cast, forged, milled or drilled through machining processes or a combination of methods in some implementations. Standard machining techniques may be used for creating or tapping the threaded holes.

In some implementations, at least a portion of the patella resurfacing implant may include a surface that is at least one of porous, textured, granular, and beaded. For example, in some implementations, the front surface of the implant and extending along all outer surfaces of the trunnions, may have a surface that may induce osseous-integration along the implant-bone interface. As noted above, the surface may include a porous, textured, granular, and/or beaded surface coating. The bearing surface may be highly polished during the manufacturing process.

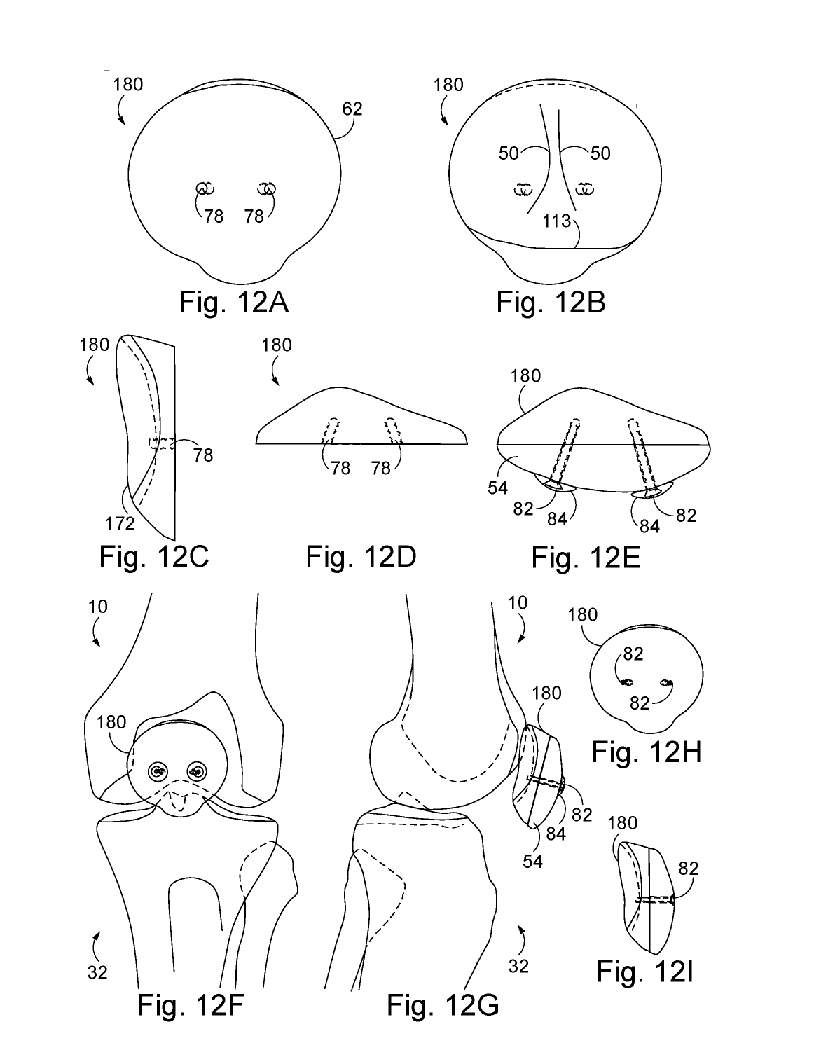

In some implementations, and referring at least to the example implementation of FIG. 12A, there is shown a front view illustrating various example aspects of a metal horizontal two-hole asymmetrical patella component 180. The prosthesis and steps for implantation may be the same or similar as that illustrated and described in example FIGS. 11A to 11I with a few example and non-limiting differences, which are described below. The example metal patella resurfacing prosthesis of may lack trunnions. In the example, it may include two of threaded hole 78 along the front of the prosthesis for screw fixation. Example FIG. 12B is a back view illustrating the asymmetrical bearing surface with articular border 113, similar in shape to the anatomical patella bearing surface and articular border. In some implementations, the prosthesis may be fully metal, including the articular bearing surface. Example FIGS. 12C and 12D are left and top views, respectively, illustrating the orientation of the two threaded holes, and asymmetrical bearing surface. Example FIGS. 12E, 12F and 12G are top, front and left views, respectively, illustrating the prosthesis implanted into a left human residual patella with two of screw 82 and washer 84 secured to both threaded holes. Example FIGS. 12H and 12I are front and left views, respectively, illustrating the prosthesis implanted into a left human residual patella with two screws without use of washers. The steps for implantation may be the same or similar as in example FIGS. 11A to 11I. In some implementations, the example and non-limiting benefits of Example FIGS. 11A to 11I may be present in the example implementation of, e.g., FIG. 12A, and may not have a need for drilling holes to accommodate trunnions into the patella as trunnions may not necessarily be included, which may simplify implantation.

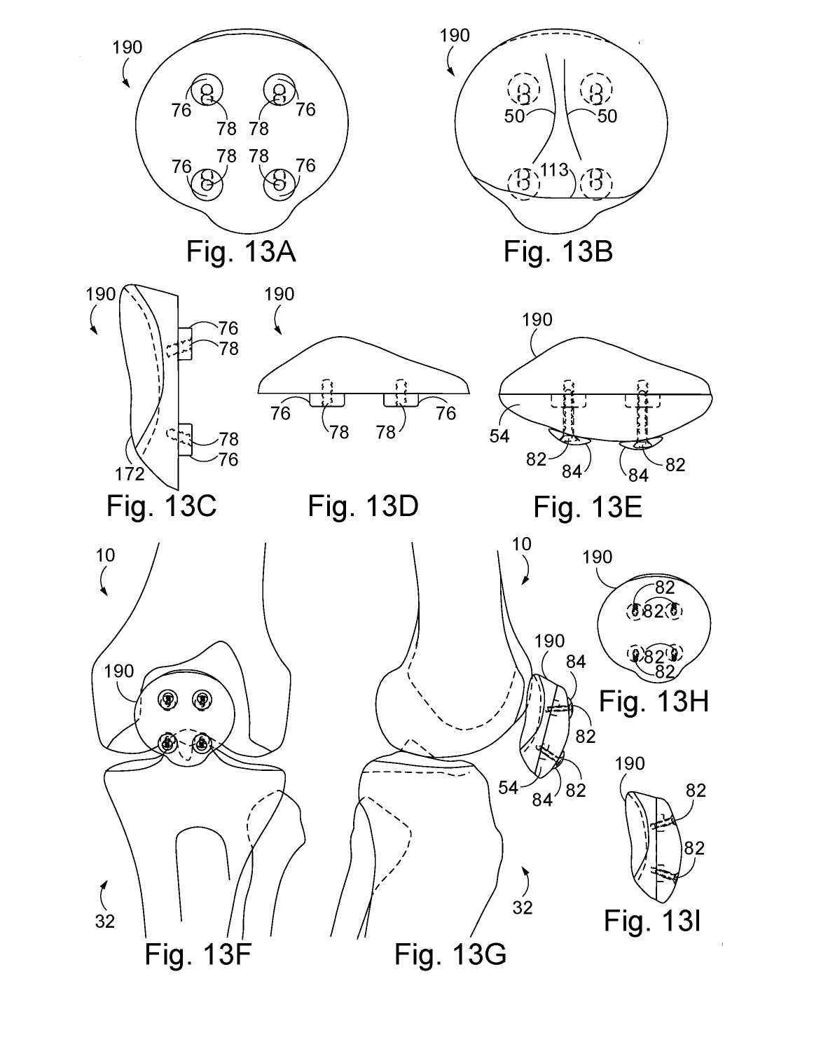

In some implementations, and referring at least to the example implementation of FIG. 13A, there is shown a front view illustrating various example aspects of a metal horizontal four-trunnion asymmetrical patella component 190. The prosthesis and steps for implantation may be the same or similar as that illustrated and described in example FIGS. 11A to 11I with a few example and non-limiting differences, which are described below. The metal patella resurfacing prosthesis may include a plurality (e.g., four) of trunnion 76 with at least one threaded hole 78 present in each. Example FIG. 13B is a back view illustrating the asymmetrical bearing surface with an articular border 113, similar in shape to the anatomical patella bearing surface and articular border. The prosthesis may be partly or fully metal, including the articular bearing surface. Example FIGS. 13C and 13D are left and top views, respectively, illustrating the arrangement of the four trunnions, orientation of the four threaded holes, and asymmetrical bearing surface. Example FIGS. 13E, 13F and 13G are top, front and left views, respectively, illustrating the prosthesis implanted into a left human residual patella with four of screw 82 and washer 84 secured to the threaded holes of all four trunnions. Example FIGS. 13H and 13I are front and left views, respectively, illustrating the prosthesis implanted into a left human residual patella with four screws without use of washers. The steps for implantation may be the same or similar as in example FIGS. 11A to 11I except four holes may be drilled into the patella for accommodation of all four trunnions along with four screw holes in alignment with the associated threaded holes of the prosthesis. In some implementations, one or more of the example and non-limiting benefits of Example FIGS. 11A to 11I may be included, but with four trunnions included, versus two, which may enable additional resistance to tensile forces provided along the medial and lateral aspects of the patella and component interface, which may increase implant stability.

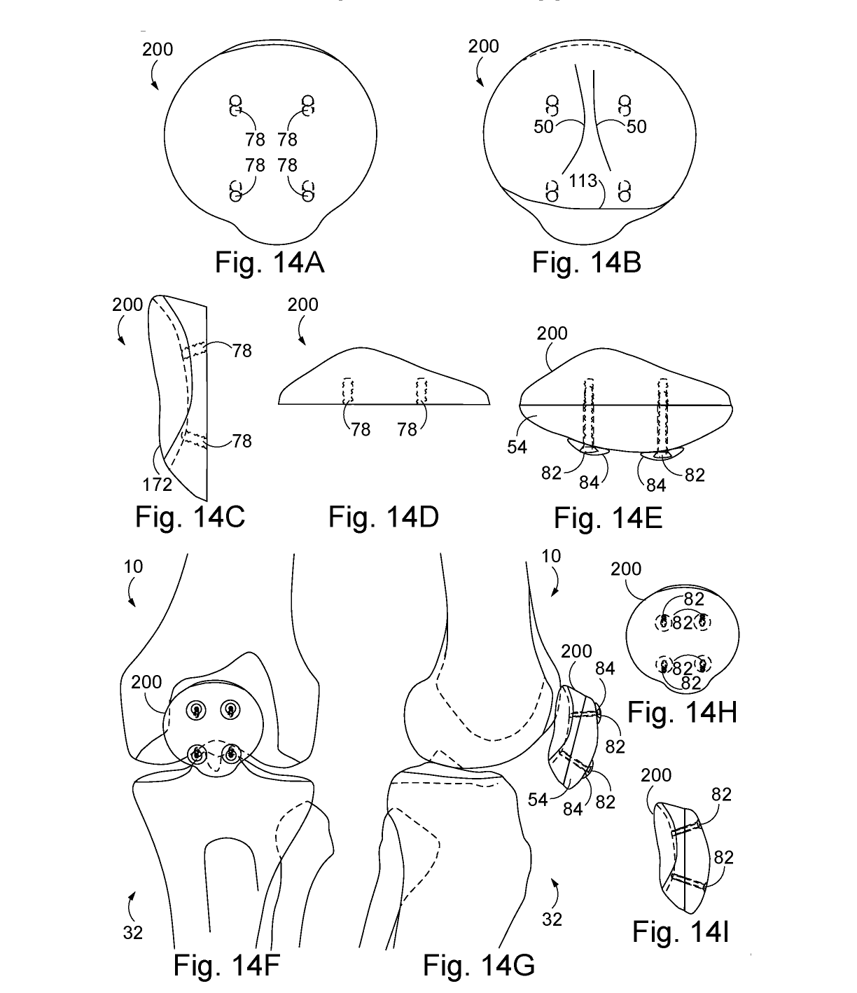

In some implementations, and referring at least to the example implementation of FIG. 14A, there is shown a front view illustrating various example aspects of a metal four-hole asymmetrical patella component 200. The prosthesis and steps for implantation may be the same or similar as that illustrated and described in example FIGS. 13A to 13I with a few example and non-limiting differences, which are described below. The metal patella resurfacing prosthesis may not include trunnions. The prosthesis may include one or more (e.g., four) of threaded hole 78. Example FIG. 14B is a back view illustrating the asymmetrical bearing surface with an articular border 113, similar in shape to the anatomical patella bearing surface and articular border. The prosthesis may be partially or fully metal, including the articular bearing surface. Example FIGS. 14C and 14D are left and top views, respectively, illustrating the example orientation of the four threaded holes, and asymmetrical bearing surface. Example FIGS. 14E, 14F and 14G are top, front and left views, respectively, illustrating the prosthesis implanted into a left human residual patella with four of screw 82 and washer 84 secured to the threaded holes. Example FIGS. 14H and 14I are front and left views, respectively, illustrating the prosthesis implanted into a left human residual patella with four screws without use of washers. The steps for implantation may be the same or similar as in example FIGS. 13A to 13I except holes may not necessarily be drilled into the patella for accommodation of trunnions. However, in some implementations, the four screw holes may be drilled in alignment with the associated threaded holes of the prosthesis. In some implementations, one or more of the example and non-limiting benefits of Example FIGS. 13A to 13I may be present, and/or may include an additional and non-limiting benefit of not requiring holes to be drilled in the patella for accommodation of the trunnions.

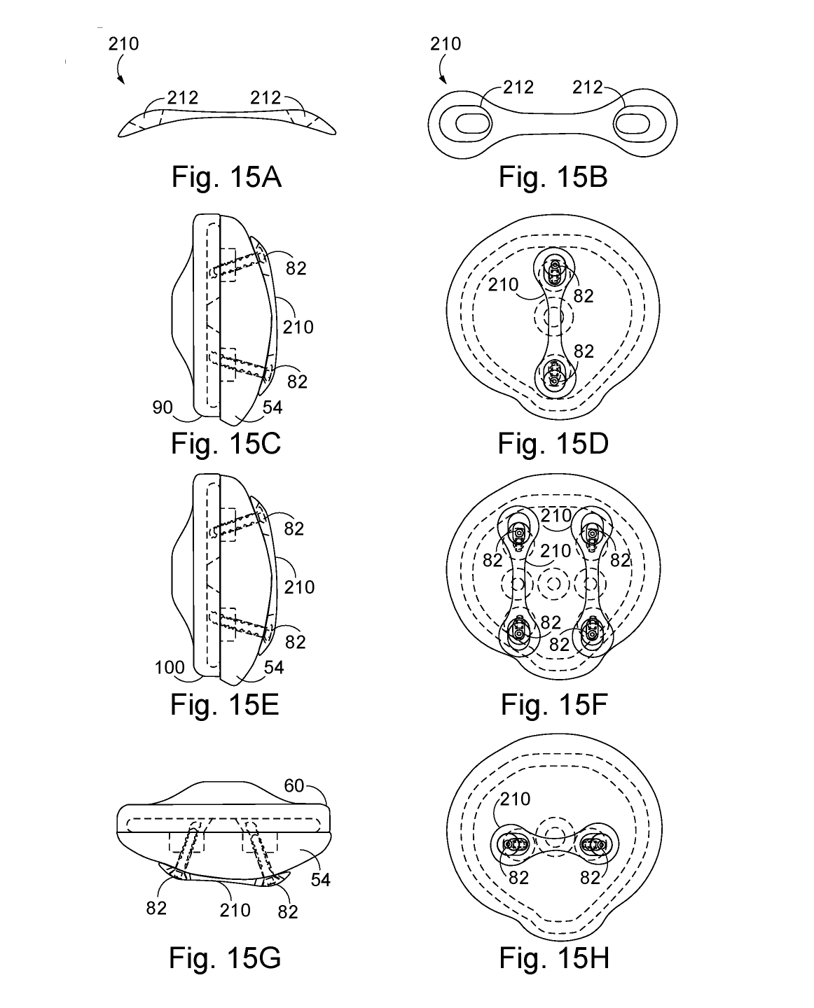

In some implementations, the patella resurfacing prosthesis may include a plate including one or more holes, the one or more holes may be configured to receive one of the one or more screws. For example, the patella resurfacing prosthesis may include a plate, configured to receive one or more screws through one or more screw holes within the plate, with the screws also engaging one or more threaded holes along the front aspect of the patella resurfacing implant or threaded holes within one or more trunnions along the front aspect of the patella resurfacing implant. For instance, in some implementations, and referring at least to the example implementation of FIGS. 15A and 15B, there is shown bottom and front views, respectively, illustrating various example aspects of plate 210 with one or more screw holes 212 for accommodation of one or more (e.g., two) screws. The surgical steps for implantation of the plate in association with the patella resurfacing prosthesis may be the same or similar as that illustrated and described in example FIGS. 2A to 2I with a few example and non-limiting additional aspects, which are described below. Example FIGS. 15C and 15D are left and front views, respectively, illustrating the vertical two-trunnion symmetrical patella component 90 implanted into a left residual patella 54 with two of screw 82 inserted through plate 210. Example FIGS. 15E and 15F are left and front views, respectively, illustrating the four-trunnion symmetrical patella component 100 implanted into a left residual patella 54 with screws inserted through each screw hole of two of plate 210. Example FIGS. 15G and 15H are top and front views illustrating the horizontal two-trunnion symmetrical patella component 60 implanted into a left residual patella 54 with two screws inserted through plate 210. The plate may be applied either singularly or in duplicate, depending on chosen implementation and application, using the same or similar surgical approach and techniques as described in example FIGS. 2A to 2I. In some implementations, after patella osteotomy and drilling of holes to accommodate associated trunnions, when present, and drilling of associated screw holes, the plate may be placed on along the anterior cortex of the patella, and the plate may undergo further contouring if needed to match the curvature of the anterior cortex of the patella. Once applied to the anterior patella cortex, each screw may then be inserted through the screw holes of the plate, through the screw holes in the residual patella and threaded and secured into the threaded holes along the front aspect of the prosthesis or the threaded holes within the trunnions along the front of the prosthesis. Use of the plate may provide for a wider surface area for stress distribution for compressive forces to further anchor the residual patella to the patella component, which may increase implant stability.

In some implementations, at least a portion of the plate may be manufactured with metal. The metal may include, e.g., a high carbon cobalt chrome (CoCr) alloy. The plate may be made of an alternate metal or metal alloy including, e.g., stainless steel, titanium, or another suitable material, including carbon fiber, plastic, etc. The plate may be formed as cast, forged, milled or drilled through machining processes or a combination of methods. Standard machining techniques and metal polishing techniques may be used.

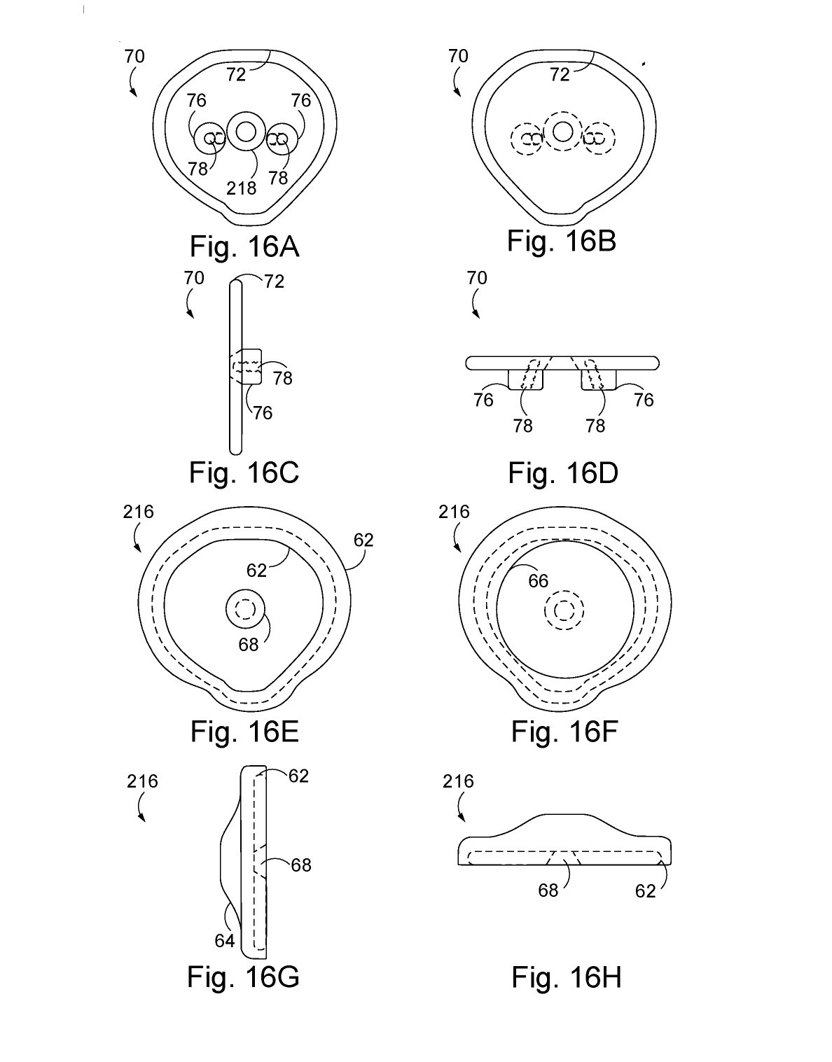

In some implementations, and referring at least to the example implementation of FIGS. 16A and 16B, there is shown front and back views, respectively, illustrating various example aspects of metal base 70 of a flush horizontal two-trunnion symmetrical patella component including two horizontally-arranged trunnions. The metal base in one or more example implementations is illustrated without the polyethylene segment and includes two of trunnion 76 each with threaded hole 78, and a flush polyethylene retainer recess 218. Example FIGS. 16C and 16D are left and top views, respectively, illustrating the metal base of the prosthesis including two horizontally-arranged trunnions. In some implementations, the one or more trunnions may be arranged along the metal base in conjunction with a symmetrical bearing surface. For instance, Example FIGS. 16E and 16F are front and back views, respectively, illustrating a polyethylene segment of the prosthesis including the flush polyethylene retainer 68. The polyethylene segment 216 is illustrated without the metal base. The polyethylene segment may include a flush polyethylene retainer 68 which may be formed during manufacturing and situated along the flush polyethylene retainer recess of the metal base. Example FIGS. 16G and 16H are left and top views further illustrating the polyethylene segment and the flush polyethylene retainer.

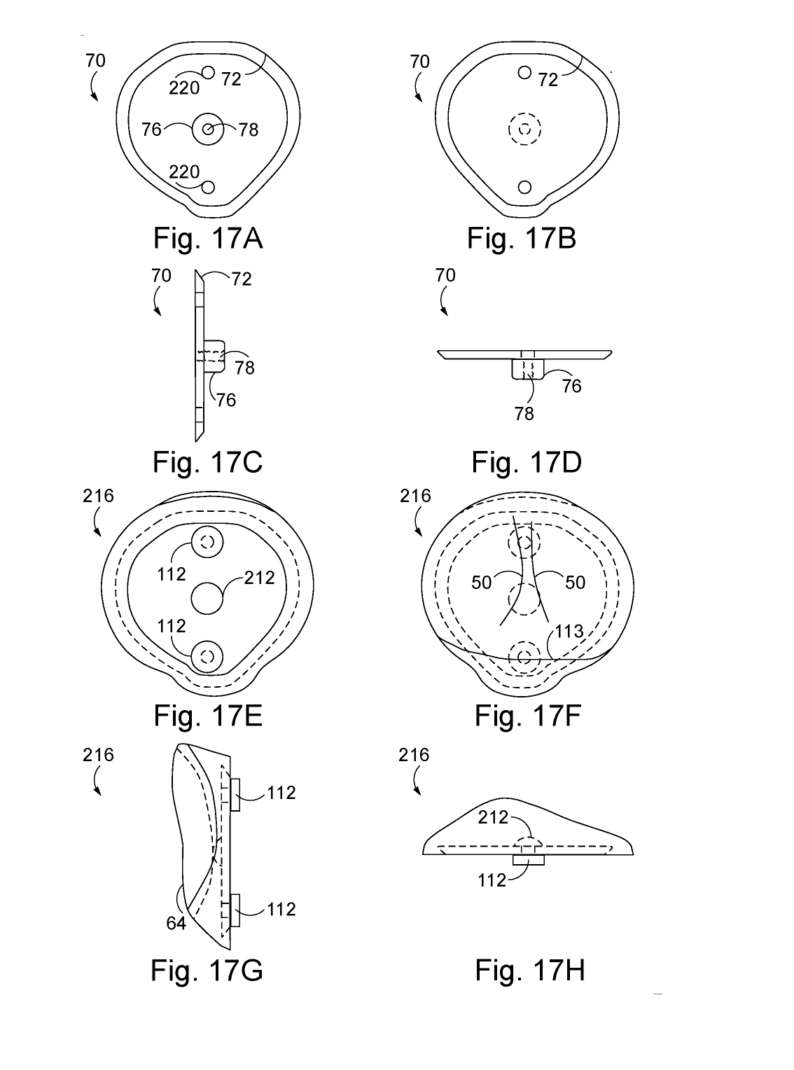

In some implementations, and referring at least to the example implementation of FIGS. 17A and 17B, there is shown front and back views, respectively, illustrating various example aspects of metal base 70 of a one-trunnion asymmetrical patella component, including one centrally-oriented trunnion. The metal base is illustrated without the polyethylene segment and may include one of trunnion 76 with a threaded hole 78, and two of non-flush polyethylene retainer hole 220. Example FIGS. 17C and 17D are left and top views, respectively, illustrating the metal base of the prosthesis including one centrally located trunnion. Example FIGS. 17E and 17F are front and back views, respectively, illustrating a polyethylene segment of the prosthesis including a centrally-oriented trunnion and an asymmetrical bearing surface with articular border 113, similar in shape to the anatomical patella bearing surface and articular border. The polyethylene segment 216 is illustrated without the metal base. The polyethylene segment may include two of non-flush polyethylene retainer 112 which may be formed during manufacturing and situated in and above the non-flush polyethylene retainer holes of the metal base, as illustrated. Example FIGS. 17G and 17H are left and top views further illustrating the polyethylene segment and the non-flush polyethylene retainers.

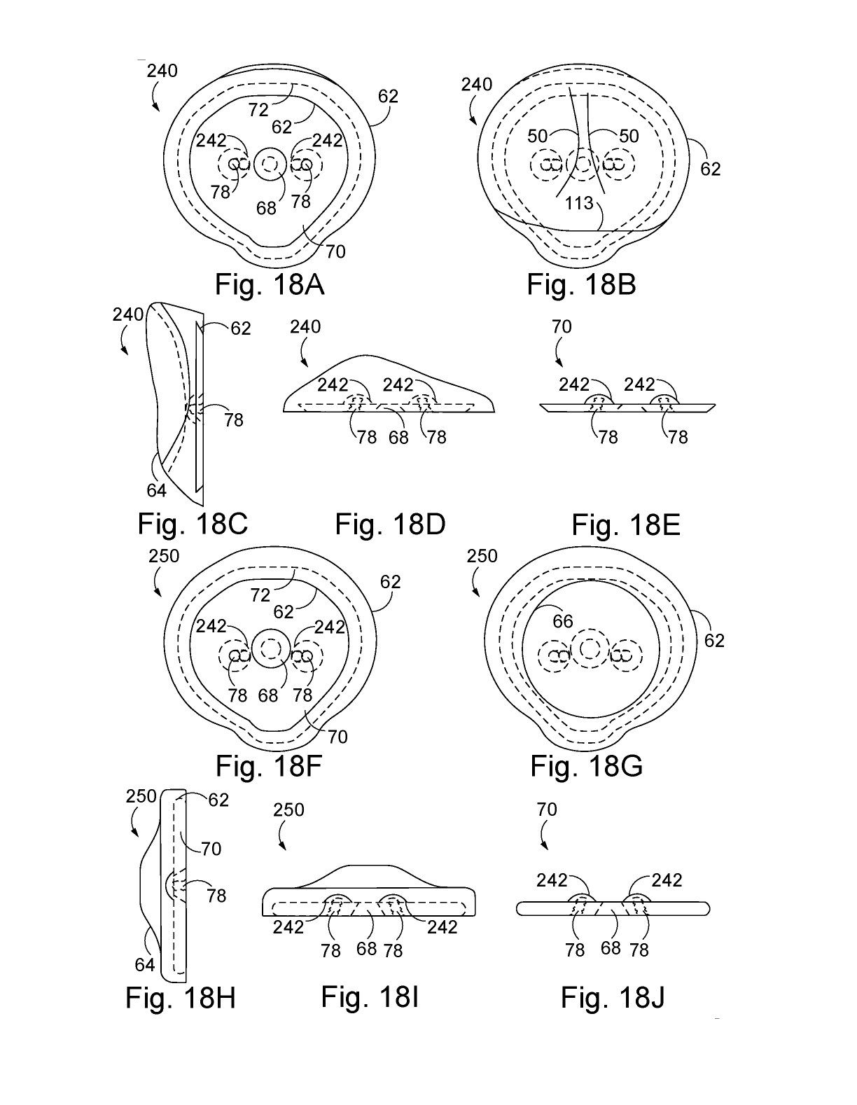

In some implementations, and referring at least to the example implementation of FIG. 18A, there is shown a front view illustrating various example aspects of a horizontal two-hole asymmetrical patella component 240. The prosthesis and steps for implantation may be the same or similar as that illustrated and described in example FIGS. 8A to 8I with a few example and non-limiting differences, which are described below. In some implementations, the prosthesis may include no trunnions, e.g., along the front of metal base 70. In some implementations, the prosthesis may include threaded holes 78 in the metal base. Example FIG. 18B is a back view illustrating the asymmetrical bearing surface with articular border 113, similar in shape to the anatomical patella bearing surface and articular border. Example FIGS. 18C and 18D are left and top views, respectively, illustrating the threaded hole 78 arrangement, flush polyethylene retainer 68, two of threaded hole extension 242, and asymmetrical bearing surface. The threaded hole extensions may allow for screws to be threaded further into the metal base. In some implementations, no holes are drilled for accommodating trunnions during implantation as no trunnions may be needed. Example FIG. 18E is a top view of the metal base 70 without the polyethylene segment further illustrating the threaded hole and associated threaded hole extensions 242.

In some implementations, and referring at least to the example implementation of FIG. 18F, there is shown a front view illustrating various example aspects of a horizontal two-hole symmetrical patella component 250. The prosthesis and steps for implantation may be the same or similar as that illustrated and described in example FIGS. 8A to 8I with a few example and non-limiting differences, which are described below. In some implementations, the prosthesis may lack trunnions along the front of the metal base 70. In some implementations, the prosthesis may include threaded holes 78 in the metal base. Example FIG. 18G is a back view illustrating the symmetrical bearing surface with a dome-shaped articular surface and the polyethylene raised segment border 66 along the periphery of its elevated aspect. Example FIGS. 18H and 18I are left and top views, respectively, illustrating the threaded hole 78 arrangement, flush polyethylene retainer 68, two of threaded hole extension 242, and symmetrical bearing surface. Example FIG. 18J is a top view of the metal base 70 without the polyethylene segment further illustrating the threaded hole and associated threaded hole extensions 242. In some implementations, no holes are drilled for accommodating trunnions during implantation as no trunnions may necessarily be present.

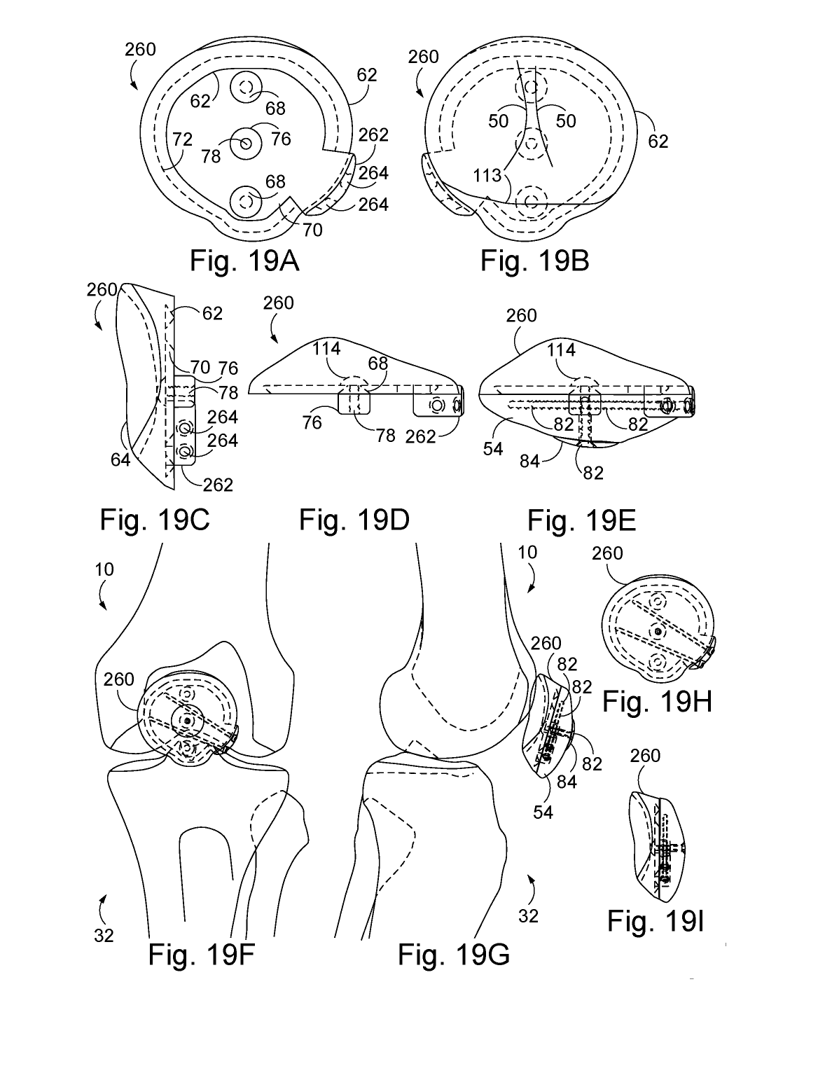

In some implementations, the patella resurfacing prosthesis may include a side-plate configured to receive one or more screws through one or more holes (e.g., one or more side-plate holes) from a periphery of the patella resurfacing implant. For instance, in some implementations, and referring at least to the example implementation of FIG. 19A, there is shown a front view illustrating various example aspects of a one-trunnion asymmetrical patella component with side-plate 260. The prosthesis and steps for implantation may be the same or similar as that illustrated and described in example FIGS. 5A to 5I with a few example and non-limiting differences, which are described below. In some implementations, the prosthesis may include, e.g., two, of flush polyethylene retainer 68 instead of the non-flush polyethylene retainers. In some implementations, the prosthesis may include side-plate 262, which may be an extension of metal base 70. This side-plate may extend beyond the outer, polyethylene periphery 62, may include, e.g., two, of side-plate hole 264, to accommodate secondary screw fixation. In some implementations, the side-plate may be located along the lower-right aspect of the prosthesis as illustrated, and the inferior-lateral aspect of the patella when implanted. In some implementations, the screw fixation from the side-plate may provide the final fixation construct with peripheral fixation, from a second plane, allowing for further stability. The side-plate may provide the ability for screw fixation into the residual patella through the periphery of the component. In some implementations, anterior fixation may be achievable through into threaded hole 78 in the associated centrally-oriented trunnion 76.

Example FIG. 19B is a back view further illustrating the polyethylene asymmetrical bearing surface with articular border 113, similar in shape to the anatomical patella bearing surface and articular border. Example FIGS. 19C and 19D are left and top views, respectively, illustrating the orientation of the central trunnion, asymmetrical bearing surface and side-plate and associated side-plate holes. Example FIGS. 19E, 19F and 19G are top, front and left views, respectively, illustrating the prosthesis implanted into a left residual patella 54 with one screw 82 and washer 84 secured to the threaded holes of the centrally-oriented trunnion. In some implementations, two screws may be secured to the residual patella through the side-plate holes directed in an inferior-lateral to superior-medial screw trajectory. Example FIGS. 19H and 19I are front and left views, respectively, illustrating the prosthesis implanted into a left residual patella with one screw without use of a washer placed secured to the centrally-oriented trunnion. Two screws may be secured through the side-plate holes from an inferior-lateral to superior-medial screw trajectory. The steps for implantation may be the same or similar as in example FIGS. 5A to 5I, although in some implementations, minor additional soft tissue attachment may be freed along the inferior-lateral aspect of the patella using a scalpel to prepare the inferior-lateral aspect of the residual patella for placement of the one-trunnion symmetrical patella component with side-plate.

In some implementations (e.g., where flush polyethylene retainers are included), there may not be a need for drilling holes for non-flush polyethylene retainers. The anterior screw fixation may be performed first with or without use of a washer. Secondary screw holes may be drilled from inferior-lateral to superior-medial, with the aid of a surgical ruler, surgical caliper and drill hole depth gauge in standard fashion, after first securing the anterior screw fixation. Care may be taken to avoid the central trunnion of the prosthesis while drilling for the side-plate screws, with one screw may be located above the central trunnion and the other below it. These two screws may be selected once the surgeon has determined the appropriate length based on the dimensions of the native patella using measurements from the surgical ruler or caliper, and drill hole depth gauge with the aid of, e.g., intraoperative fluoroscopic imaging, and these screws may then be tightened and secured. In some implementations, the prosthesis fixation may be two-plane in nature with both anterior to posterior fixation afforded by screw fixation to the centrally-arranged trunnion and inferior-lateral to superior-medial fixation afforded by screw fixation through the side-plate holes. This fixation mode may provide two plane fixation and increased implant stability for use in the setting of diminished bone quality.

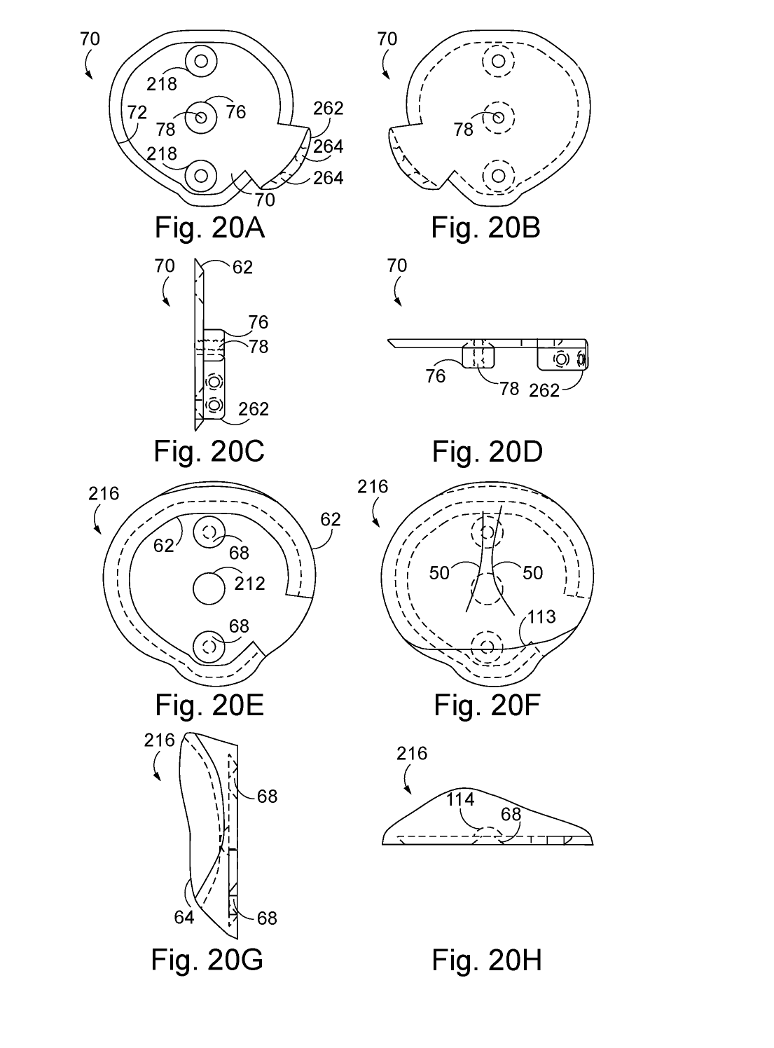

In some implementations, and referring at least to the example implementation of FIGS. 20A and 20B, there is shown front and back views, respectively, illustrating various example aspects of metal base 70 of a one-trunnion asymmetrical patella component with side-plate. The metal base is illustrated without the polyethylene segment and may include one of trunnion 76 with threaded hole 78, and two of flush polyethylene retainer recess 218. The metal base may include side-plate 262 with two of side-plate hole 264. Example FIGS. 20C and 20D are left and top views, respectively, further illustrating the metal base of the prosthesis including one centrally located trunnion, the side-plate and its associated side-plate holes. Example FIGS. 20E and 20F are front and back views, respectively, illustrating a polyethylene segment of the prosthesis including a centrally-oriented trunnion and an asymmetrical bearing surface with articular border 113, similar in shape to the anatomical patella bearing surface and articular border. The polyethylene segment 216 is illustrated without the metal base. The polyethylene may be formed to accommodate the metal base, including the side-plate. The polyethylene segment may include two of flush polyethylene retainer 68, which may be formed during manufacturing around the metal base, and situated in and above the flush polyethylene retainer recesses of the metal base. Example FIGS. 20G and 20H are left and top views further illustrating the polyethylene segment and the flush polyethylene retainers.

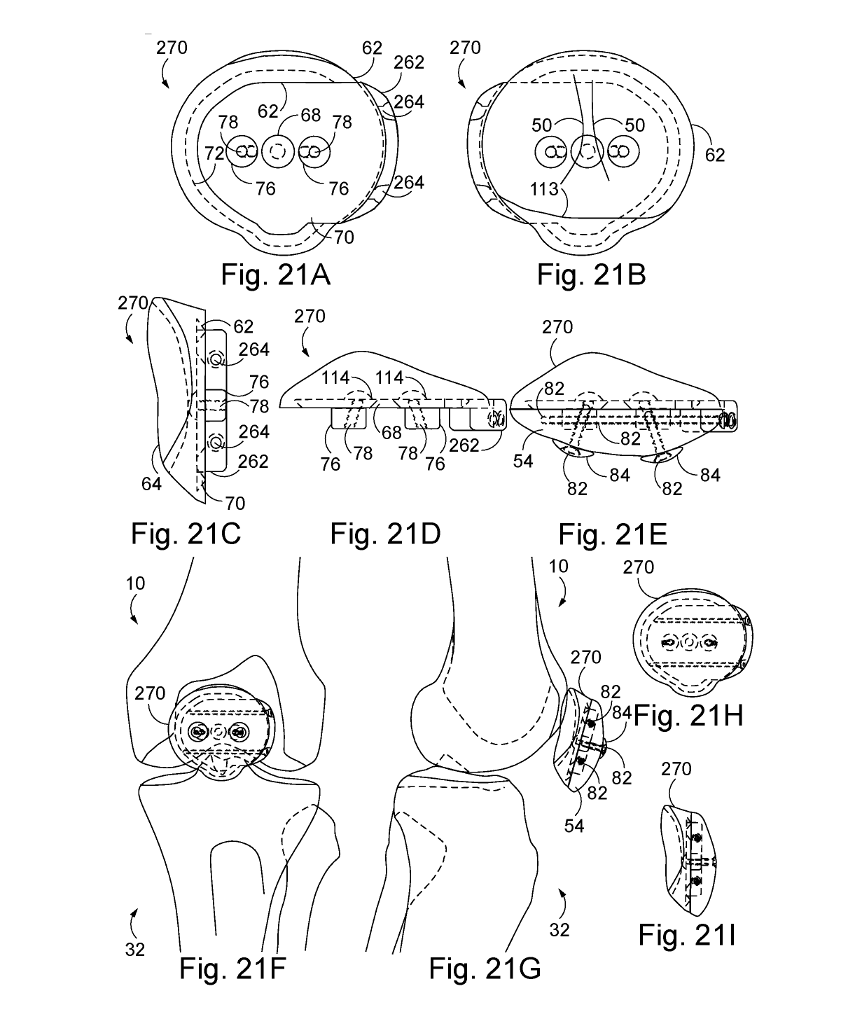

In some implementations, and referring at least to the example implementation of FIG. 21A, there is shown a front view illustrating various example aspects of a flush horizontal two-trunnion asymmetrical patella component with side-plate 270. The prosthesis and steps for implantation may be the same or similar as that illustrated and described in example FIGS. 19A to 19I with a few example and non-limiting differences, which are described below. In some implementations, the prosthesis may have one flush polyethylene retainer 68. The prosthesis may include a side-plate 262, which may be an extension of metal base 70. This side-plate may extend beyond the outer, polyethylene periphery 62, and may include two of side-plate hole 264, to accommodate secondary screw fixation. The side-plate may be located along the right aspect of the prosthesis as illustrated, and the lateral aspect of the patella when implanted. Anterior fixation may be achievable using, e.g., two screws secured into each threaded hole 78 of each trunnion 76. The side-plate may provide the ability for screw fixation into the residual patella from the periphery of the component. Example FIG. 21B shows a back view further illustrating the polyethylene asymmetrical bearing surface with an articular border 113, similar in shape to the anatomical patella bearing surface and articular border.

In some implementations, the side-plate may be situated along the right aspect of the prosthesis, allowing screws placement through the side-plate holes to be directed in a horizontal trajectory, above and below the (two) trunnions. FIGS. 21C and 21D show left and top views, respectively, illustrating the orientation of the horizontally-arranged trunnions, asymmetrical bearing surface and side-plate and associated side-plate holes. Example FIGS. 21E, 21F and 21G are top, front and left views, respectively, illustrating the prosthesis implanted into a left human residual patella 54 with two of screw 82 and two of washer 84 secured to the threaded holes of the two trunnions. In some implementations, two screws may be secured through the side-plate holes in a lateral to medial screw trajectory. Example FIGS. 21H and 21I are front and left views, respectively, illustrating the prosthesis implanted into a left human residual patella with two screws without washers placed, secured to both trunnions. In some implementations, two screws may secured into to the residual patella through the side-plate holes in a lateral to medial screw trajectory. The steps for implantation may be the same or similar as in example FIGS. 19A to 19I except in some implementations may include minor additional soft tissue attachment to be freed along the inferior-lateral aspect of the patella using, e.g., a scalpel to prepare the residual patella for placement of the prosthesis with its associated side-plate. The secondary screw holes may be drilled from lateral to medial, with the aid of, e.g., a surgical ruler, surgical caliper and drill hole depth gauge, after first securing the anterior screw fixation. Care may be taken to avoid the two, horizontally-arranged, trunnions of the prosthesis while pre-drilling for the placement of side-plate screws, with one screw that may be located above the trunnions and the other below them. These two screws may be selected once the surgeon has determined the appropriate length based on the dimensions of the native patella using measurements from the surgical ruler or caliper, and drill hole depth gauge with the aid of, e.g., intraoperative fluoroscopic imaging and these screws may then be tightened and secured. In some implementations, the prosthesis fixation may be two-plane in nature with both anterior to posterior fixation afforded by screw fixation to the two horizontally-arranged trunnions and lateral to medial fixation afforded by screw fixation through the side-plate holes. This fixation mode may provide rigid fixation strength for use in the setting of diminished bone quality and with additional two-plane resistance to tensile forces provided along the medial and lateral aspects of the patella and component interface, which may increase implant stability in the setting of diminished bone quality.

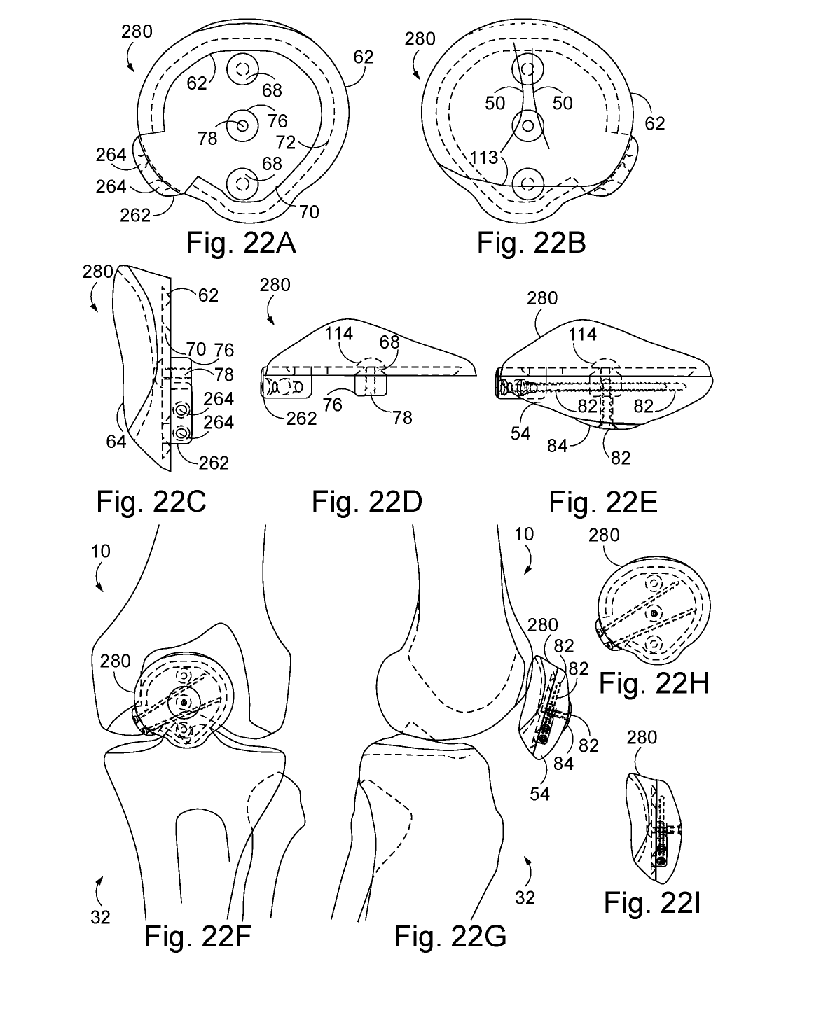

In some implementations, and referring at least to the example implementation of FIG. 22A, there is shown a front view illustrating various example aspects of flush one-trunnion asymmetrical patella component with side-plate 280. The prosthesis and steps for implantation may be the same or similar as that illustrated and described in example FIGS. 19A to 19I with a few example and non-limiting differences, which are described below. The prosthesis may include side-plate 262, which may be an extension of metal base 70. This side-plate may extend beyond the outer, polyethylene periphery 62, and may include, e.g., two, of side-plate hole 264, to accommodate secondary screw fixation. In some implementations, the side-plate may be located along the lower-left aspect of the prosthesis as illustrated, and the inferior-medial aspect of the patella when implanted. The side-plate may provide the ability for screw fixation into the residual patella from the periphery of the component.

Example FIG. 22B is a back view further illustrating the polyethylene asymmetrical bearing surface with articular border 113, similar in shape to the anatomical patella bearing surface and articular border. Example FIGS. 22C and 22D are left and top views, respectively, illustrating the orientation of the central trunnion, asymmetrical bearing surface and side-plate and associated side-plate holes. Example FIGS. 22E, 22F and 22G are top, front and left views, respectively, illustrating the prosthesis implanted into a left residual patella 54 with, e.g., one screw 82 and washer 84 secured to the threaded holes of the centrally-oriented trunnion. In some implementations, two screws may be secured to the residual patella through the side-plate holes in an inferior-medial to superior-lateral screw trajectory. Example FIGS. 22H and 22I are front and left views, respectively, illustrating the prosthesis implanted into a left residual patella with one screw without use of a washer placed secured to the centrally-oriented trunnion. Two screws may be secured to the residual patella through the side-plate holes in an inferior-medial to superior-lateral trajectory. The steps for implantation may be the same or similar as in example FIGS. 19A to 19I except that the surgical approach utilized may be a medial parapatellar approach with a medial arthrotomy and dissection with patella inversion through the same medial arthrotomy. Minor additional soft tissue attachment may be freed along the inferior-medial aspect of the patella using, e.g., a scalpel to prepare the residual patella for placement of the prosthesis with its associated side-plate. The anterior screw fixation may be performed first with or without use of a washer. Secondary side-plate screw holes may be drilled from inferior-medial to superior-lateral, with the aid of, e.g., a surgical ruler, surgical caliper and drill hole depth gauge with the aid of, e.g., intraoperative fluoroscopic imaging in standard fashion, after first securing the anterior screw fixation. In some implementations, e.g., with the side-plate arranged along the lower-left side of the implant and the inferior-medial aspect of the patella when implanted, a fully medial parapatellar surgical approach may be utilized during implantation. In some implementations, the prosthesis fixation may be two-plane in nature with both anterior to posterior fixation afforded by screw fixation to the centrally-arranged trunnion and inferior-medial to superior-lateral fixation afforded by screw fixation through the side-plate holes. This fixation mode may provide resistance to tensile forces provided along the medial and lateral aspects of the patella and greater fixation strength in the setting of diminished bone quality.

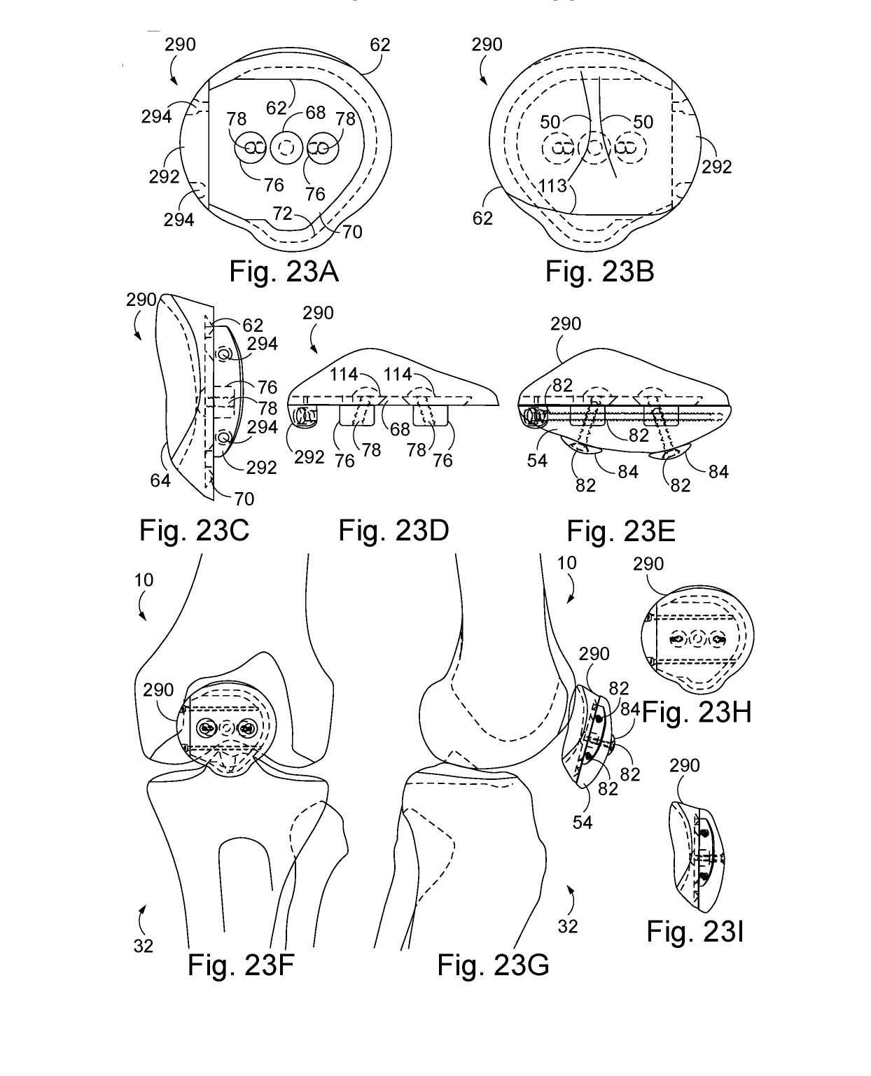

In some implementations, and referring at least to the example implementation of FIG. 23A, there is shown a front view illustrating various example aspects of a horizontal two-trunnion asymmetrical patella component with integrated side-plate 290. The prosthesis and steps for implantation may be the same or similar as that illustrated and described in example FIGS. 21A to 21I with a few example and non-limiting differences, which are described below. In some implementations, there may be included one or more flush polyethylene retainer 68. In some implementations, an integrated side-plate 292 on the left side of the prosthesis may be included, which may be an extension of metal base 70. This integrated side-plate need not extend beyond the outer border of polyethylene periphery 62, and may include two of integrated side-plate hole 294 to accommodate peripheral screw fixation. The integrated side-plate may be located along the left aspect of the prosthesis as illustrated, and the medial aspect of the patella when implanted. Anterior fixation may be achievable using, e.g., two, screws secured into each threaded hole 78 of each trunnion 76. The integrated side-plate may provide the ability for screw fixation into the prosthesis from the periphery of the patella.

Example FIG. 23B is a back view further illustrating the polyethylene asymmetrical bearing surface with an articular border 113, similar in shape to the anatomical patella bearing surface and articular border. Example FIGS. 23C and 23D are left and top views, respectively, illustrating the orientation of the horizontally-arranged trunnions, asymmetrical bearing surface and integrated side-plate and its integrated side-plate holes. Example FIGS. 23E, 23F and 23G are top, front and left views, respectively, illustrating the prosthesis implanted into a left residual patella 54 with two of screw 82 and two of washer 84 secured to the threaded holes of the two trunnions. In some implementations, two screws may be secured to the residual patella through the integrated side-plate holes in a medial to lateral screw trajectory. Example FIGS. 23H and 23I are front and left views, respectively, illustrating the prosthesis implanted into a left residual patella with two screws without washers placed, secured to both trunnions. In some implementations, two screws may be secured to the residual patella through the integrated side-plate holes in a medial to lateral screw trajectory. The steps for implantation may be the same or similar as in example FIGS. 21A to 21I except that the surgical approach may utilize a medial parapatellar approach with a medial arthrotomy and dissection with patella inversion through the same medial arthrotomy. Minor additional soft tissue attachment may be freed along the medial aspect of the patella using, e.g., a scalpel to prepare the residual patella for placement of the prosthesis with its integrated side-plate. In some implementations, a vertical osteotomy may be carried out along the medial margin of the patella. This may be performed by first measuring from the medial border of the patella, a segment of the medial margin of the patella to be removed which may match the selected width of the integrated side-plate for the chosen prosthesis. A surgical marker may be used to demarcate the planned vertical osteotomy of the medial border of the patella. K-wires may be implanted vertically along the planned osteotomy border into the anterior cortex of the patella to mark the planned osteotomy and to confirm the osteotomy alignment fluoroscopically. The k-wires may be removed leaving small k-wire holes in the anterior patella cortex, a flat bladed retractor may be placed between the patella and distal femur to protect the articular surface of the femur while stabilizing the patella with surgical clamps. An oscillating surgical bone saw may then be used to perform this osteotomy.

In some implementations, the secondary screw holes may be drilled from medial to lateral, with the aid of, e.g., a surgical ruler, surgical caliper and drill hole depth gauge, after first securing the anterior screw fixation. Care may be taken to avoid the horizontally-arranged trunnions of the prosthesis while pre-drilling for placement of the integrated side-plate screws, with one screw that may be located above the trunnions and the other below them. These two screws may be selected once the surgeon has determined the appropriate length based on the dimensions of the native patella using measurements from, e.g., the surgical ruler or caliper and drill hole depth gauge with the aid of, e.g., intraoperative fluoroscopic imaging and these screws may then be tightened and secured to the residual patella. In some implementations, the integrated side-plate need not extend beyond the polyethylene periphery the prosthesis and may maintain a more anatomic shape. In some implementations, a side-plate may be configured to receive one of the one or more screws through one or more side-plate holes from a periphery of the patella resurfacing implant. For example, in some implementations, the prosthesis fixation may be two-plane in nature with both anterior to posterior fixation afforded by screw fixation to the two horizontally-arranged trunnions and medial to lateral fixation afforded by screw fixation through the side-plate holes. This fixation mode may provide rigid fixation strength for use in the setting of diminished bone quality and with additional resistance to tensile forces provided along the medial and lateral aspects of the patella and greater fixation strength without excessive side-plate protrusion beyond the periphery of the patella.

In some implementations, the side-plate may include a symmetrical bearing surface. For example, in some implementations, and referring at least to the example implementation of FIG. 24A, there is shown a front view illustrating various example aspects of flush one-trunnion symmetrical patella component with side-plate 300. The prosthesis and steps for implantation may be the same or similar as that illustrated and described in example FIGS. 19A to 19I with a few example and non-limiting differences, which are described below. The prosthesis may include side-plate 262, which may be an extension of metal base 70. This side-plate may extend beyond the outer, polyethylene periphery 62, and may include, e.g., two, of side-plate hole 264, to accommodate secondary screw fixation. In some implementations, the side-plate may be located along the lower-right aspect of the prosthesis as illustrated, and the inferior-lateral aspect of the patella when implanted. The side-plate may provide the ability for screw fixation into the residual patella from the periphery of the component.

Example FIG. 24B is a back view further illustrating the dome-shaped, polyethylene symmetrical bearing surface, with the polyethylene raised segment border 66. Example FIGS. 24C and 24D are left and top views, respectively, illustrating the orientation of the central trunnion, symmetrical bearing surface and side-plate and associated side-plate holes. Example FIGS. 24E, 24F and 24G are top, front and left views, respectively, illustrating the prosthesis implanted into a left residual patella 54 with, e.g., one screw 82 and washer 84 secured to the threaded holes of the centrally-oriented trunnion. In some implementations, two screws may be secured to the residual patella through the side-plate holes in an inferior-lateral to superior-medial screw trajectory. Example FIGS. 24H and 24I are front and left views, respectively, illustrating the prosthesis implanted into a left residual patella with one screw without use of a washer placed secured to the centrally-oriented trunnion. Two screws may be secured to the residual patella through the side-plate holes in an inferior-lateral to superior-medial screw trajectory. The steps for implantation may be the same or similar as in example FIGS. 19A to 19I. In some implementations, the prosthesis fixation may be two-plane in nature with both anterior to posterior fixation afforded by screw fixation to the centrally-arranged trunnion and inferior-lateral to superior-medial fixation afforded by screw fixation through the side-plate holes. This fixation mode may provide resistance to tensile forces provided along the medial and lateral aspects of the patella and greater fixation strength in the setting of diminished bone quality.

In some implementations, the patella resurfacing prosthesis may provide for both un-cemented and cemented applications, and for options for multiple fixation modes. In some implementations, the prosthesis may be implanted in the same or similar surgical setting and in combination with a femoral trochlear component. An anatomical, asymmetrical bearing surface or non-anatomical, dome-shaped bearing surface prosthesis implementation may be selected for implantation with a choice of femoral trochlear component selected accordingly. In some implementations, additional construct support may be achievable through use of a prosthesis with, e.g., additional trunnions and their associated threaded holes, supplemental anterior plate application, and/or through an implementation with the above-noted side-plate. The side-plate may provide the ability for screw fixation into the prosthesis from the periphery of the patella. Through selection of various example implementations, construct support options may be selected based on individualized need for fixation and stability also allowing the surgeon to address bone deficiency.

It will be appreciated that the patella resurfacing prosthesis implementations may have both a left knee and right knee version. Although a left knee version is shown in example figures, the right-sided patella resurfacing prosthesis version may be essentially a mirror image of the left-sided version. This mirror image may apply to the back of the prosthesis, articular bearing surface, and to the front, along which the threaded holes and/or trunnions (when applicable) may situated as well as the side-plates, peripheral or integrated.

It will also be appreciated that while one or more example implementations are described with specific details, this should not be construed as limitations on the scope of the disclosure. For instance, multiple additional configurations are contemplated for the patella resurfacing prosthesis. For example, inclusion of a different number of trunnions (or no trunnions) in some implementations may be possible, and may include, e.g., one, two, three, four, or more trunnions. As yet another example, inclusion of a different number of screws (or no screws) in some implementations may be possible, and may include, e.g., one, two, three, four, or more screws (with or without the application of bone cement during implantation).

As another example, the plate of example FIGS. 15A to 15H may have an increased or decreased number of screw holes to accommodate a different number of screws. The plates may be wider or more narrow in order to accommodate a different number of holes or altered placement along the surface of the patella. As yet another example, the plate may include a screw holes or multiple screw holes with threading along the periphery of the screw hole(s). These locking screw holes may be designed to engage screws with threading along the outer screw head (e.g., locking screws). This may allow for screws, with corresponding added threading along the outer screw head, to engage the periphery of the screw holes and thereby thread into the screw holes of the plate, thus allowing for secondary fixation of the screws to the plate and residual patella. A combination of threaded and unthreaded screw holes may be present in the plate.

As yet another example, in some implementations, the side-plate of Example FIGS. 19A to 19I, 21A to 21I, 22A to 22I, and 23A to 23I may include a side-plate hole or multiple side-plate holes with threading along the periphery of the screw hole(s) designed to engage screws with threading along the outer screw head which are known in the art as locking screws. This would effectively allow for screws, with corresponding added threading along the outer screw head, to engage the periphery of screw holes and thereby thread into the side-plate holes, thus allowing for secondary fixation of the screws to the side-plate and residual patella. A combination of threaded and unthreaded screw holes may be present in the plate. As another example, in some implementations, the side-plate illustrated in example FIG. 19A may be located at the top-left or top-right side prosthesis, to achieve a screw trajectory when implanted of superior-medial to inferior-lateral or superior-lateral to inferior-medial, respectively. As yet another example, in some implementations, the side-plate illustrated in example FIG. 21A may be located along the left side of the prosthesis, to achieve a screw trajectory when implanted of medial to lateral. As yet another example, in some implementations, the side-plate of Example FIGS. 19A to 19I, 21A to 21I, 22A to 22I, and 23A to 23I may include a different number of side-plate holes including 1, 3, 4 or more and may be wider or narrower to accommodate a higher or lower number of side-plate holes, respectively. As yet another example, in some implementations, the integrated side-plate of Example FIG. 23A may be located along the right side of the prosthesis so that the screw trajectory when implanted may be lateral to medial instead of medial to lateral. This may position the integrated side-plate along the lateral side of the patella when implanted and may require a vertical osteotomy of the lateral margin of the patella to accommodate the integrated side-plate of the prosthesis.

As yet another example, in some implementations, the side-plates may be combined so that the prosthesis includes more than one side-plate, thereby accommodating additional peripheral screws for implant fixation. For instance, both side-plates illustrated in example FIGS. 19A to 19I and Example FIGS. 22A to 22I may be present in one patella resurfacing prosthesis with shorter screws used in side-plate holes to allow peripheral screw fixation from the inferior-lateral and inferior-medial aspects of the patella. Dual integrated side-plates may be possible, whereby the left-sided integrated side-plate in example FIGS. 23A to 23I may be present in implementations with an integrated side-plate along the right side of the prosthesis.

As yet another example, in some implementations, the side-plate or integrated side-plate may be taller so that the side-plate extends further towards the anterior cortex of the patella when implanted and/or the side-plate holes may be raised higher so that screws can be closer to the anterior cortex of the patella when implanted. In some implementations, the converse may be possible with a shorter side-plate or shorter integrated side-plate. The side-plate holes may be angled to allow screws placed in the side-plate holes to be angled to the anterior cortex of the anterior cortex. As yet another example, in some implementations, inclusion of the side-plate may be possible with a combination of a different number of trunnions, for instance one, two, three, four, or more trunnions, or even no trunnions with presence of only threaded holes, allowing for two-plane fixation to still be achieved.

As yet another example, in some implementations, such as those with a porous undersurface along the front aspect of the implant, the anterior screw fixation and/or peripheral screw fixation may be removed if desired after implant stability corresponding to osseous-integration has been achieved although screw fixation may still provide additional fixation support in the setting of osseous-integration, which may be beneficial to maintain.

As yet another example, in some implementations, the patella resurfacing prosthesis, screws, and/or plates may each be made of metal or metal alloy, including, e.g., high carbon cobalt chrome (CoCr) alloy, stainless steel, titanium or aluminum, ceramic-coated metal, oxidized metals, polyethylene, or another suitable material, or combination of suitable materials. They may be manufactured as cast or using additional techniques including, e.g., injection molding, forging, bending during the machining process, and may be manufactured in segments and welded, plastic welded or otherwise joined, e.g., with an inference fit. As such, the description of the example implementations for the patella resurfacing prosthesis should be used as example only and not to otherwise limit the scope of the disclosure.

While one or more example implementations have been described using a patella resurfacing prosthesis, it will be appreciated that the present disclosure may be adapted for use for a prosthesis other than a patella resurfacing prosthesis. As such, the description of a patella resurfacing prosthesis should be used as example only and not to otherwise limit the scope of the disclosure.

It will be appreciated that although the present disclosure has been described with reference to particular materials, manufacturing methods and joining methods, other suitable materials such as, for example, alternate plastics, other manufacturing methods such as, for example, injection molding, laser cutting or alternate machining methods, and other joining methods such as, for example, friction fitting may be used. Moreover, while example implementations have been described with specific features/arrangements of features, the features described in any of the implementations may be used in any combination (e.g., number of screws and their various locations, number of trunnions and their various locations, number of side-plate(s) and their various locations, no side plate(s), symmetrical bearing surfaces for any of the components, asymmetrical bearing surfaces for any of the components, horizontal arrangements, vertical arrangements, number of washers, no washers, channels and their various locations, no channels, porous or non-porous surfaces, etc.).

While one or more example implementations have been described using a patella resurfacing prosthesis for a human, it will be appreciated that the present disclosure may be adapted for use for animals as well. As such, the description of a patella resurfacing prosthesis for a human should be used as example only and not to otherwise limit the scope of the disclosure.

The terminology used herein is for the purpose of describing particular implementations only and is not intended to be limiting of the disclosure. As used herein, the singular forms “a”, “an” and “the” are intended to include the plural forms as well, unless the context clearly indicates otherwise. As used herein, the language “at least one of A, B, and C” (and the like) should be interpreted as covering only A, only B, only C, or any combination of the three, unless the context clearly indicates otherwise. It will be further understood that the terms “comprises” and/or “comprising,” when used in this specification, specify the presence of stated features, integers, steps (not necessarily in a particular order), operations, elements, and/or components, but do not preclude the presence or addition of one or more other features, integers, steps (not necessarily in a particular order), operations, elements, components, and/or groups thereof.

The corresponding structures, materials, acts, and equivalents (e.g., of all means or step plus function elements) that may be in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications, variations, substitutions, and any combinations thereof will be apparent to those of ordinary skill in the art without departing from the scope and spirit of the disclosure. The implementation(s) were chosen and described in order to explain the principles of the disclosure and the practical application, and to enable others of ordinary skill in the art to understand the disclosure for various implementation(s) with various modifications and/or any combinations of implementation(s) as are suited to the particular use contemplated.

Having thus described the disclosure of the present application in detail and by reference to implementation(s) thereof, it will be apparent that modifications, variations, and any combinations of implementation(s) (including any modifications, variations, substitutions, and combinations thereof) are possible without departing from the scope of the disclosure defined in the appended claims.

What is claimed is:

1. A patella resurfacing prosthesis comprising:
a patella resurfacing implant; and
one or more threaded holes along a front aspect of the patella resurfacing implant, wherein the one or more threaded holes along the front aspect of the patella resurfacing implant are configured to fixate the patella resurfacing implant to a patella of a user.

2. The patella resurfacing prosthesis of claim 1 further comprising one or more trunnions, wherein the one or more threaded holes along the front aspect of the patella resurfacing implant are within the one or more trunnions respectively.

3. The patella resurfacing prosthesis of claim 2 wherein the one or more trunnions include two or more trunnion arranged horizontally.

4. The patella resurfacing prosthesis of claim 2 wherein the one or more trunnions include two or more trunnions arranged vertically.

5. The patella resurfacing prosthesis of claim 2 wherein at least one trunnion of the one or more trunnions is a central trunnion.

6. The patella resurfacing prosthesis of claim 2 wherein the one or more trunnions are arranged along with an asymmetrical bearing surface.

7. The patella resurfacing prosthesis of claim 2 wherein the one or more trunnions are arranged along with a symmetrical bearing surface.

8. The patella resurfacing prosthesis of claim 2 wherein the one or more trunnions are configured to receive one or more screws with one or more washers.

9. The patella resurfacing prosthesis of claim 8 further comprising a plate including one or more screw holes, the one or more screw holes configured to receive one of the one or more screws.

10. The patella resurfacing prosthesis of claim 8 further comprising a side-plate configured to receive one of the one or more screws through one or more side-plate holes from a periphery of the patella resurfacing implant.

11. The patella resurfacing prosthesis of claim 1 further comprising a base, wherein the one or more threaded holes along the front aspect of the patella resurfacing implant is within the base.

12. The patella resurfacing prosthesis of claim 11 wherein the base includes a metal base.

13. The patella resurfacing prosthesis of claim 11 wherein the one or more threaded holes are configured to receive one or more screws with one or more washers.

14. The patella resurfacing prosthesis of claim 1 further comprising a plate, wherein the plate is configured to receive one or more screws through one or more screw holes within the plate, with the screws configured to engage the threaded holes along the front aspect of the patella resurfacing implant.

15. The patella resurfacing prosthesis of claim 14 further comprising one or more screw holes within the plate configured to receive one or more screws.

16. The patella resurfacing prosthesis of claim 1 further comprising one or more channels configured to receive bone cement.

17. The patella resurfacing prosthesis of claim 1 further comprising a polyethylene segment.

18. The patella resurfacing prosthesis of claim 1 further comprising one or more side-plate holes along a periphery of the patella resurfacing implant.

19. The patella resurfacing prosthesis of claim 18 further comprising a side-plate configured to receive one or more screws through the one or more side-plate holes from a periphery of the patella resurfacing implant.

20. The patella resurfacing prosthesis of claim 18 wherein a bearing surface of the patella resurfacing implant includes an asymmetrical bearing surface.

21. The patella resurfacing prosthesis of claim 18 wherein a bearing surface of the patella resurfacing implant includes a symmetrical bearing surface.

22. The patella resurfacing prosthesis of claim 1 wherein at least a portion of the patella resurfacing implant includes a surface that is at least one of porous, textured, granular, and beaded.

23. The patella resurfacing prosthesis of claim 2 wherein the one or more trunnions are configured to receive one or more screws without one or more washers.

24. The patella resurfacing prosthesis of claim 1 wherein the one or more threaded holes are arranged along with an asymmetrical bearing surface.

25. The patella resurfacing prosthesis of claim 1 wherein the one or more threaded holes are arranged along with a symmetrical bearing surface.
